(19) United States
(12) Patent Application Publication
(10) Pub. No.: US 2016/0287534 A1
(43) Pub. Date: Oct. 6, 2016

(54) METHODS FOR REVERSING FIBROSIS

(71) Applicant: Ji-Young Lee, Coventry, CT (US)

(72) Inventor: Ji-Young Lee, Coventry, CT (US)

(73) Assignee: The University of Connecticut, Farmington, CT (US)

(21) Appl. No.: 14/676,624

(22) Filed: Apr. 1, 2015

(51) Int. Cl. A61K 31/122

(52) U.S. Cl. CPC A61K 31/122

(57) ABSTRACT

The present invention provides methods of reversing the activation of hepatic stellate cells using astaxanthin (ASTX), or a pharmaceutically acceptable salt thereof, for use in the reversal of fibrosis and fibrotic diseases.

### STATEMENT REGARDING FEDERALLY SPONSORED RESEARCH OR DEVELOPMENT

This invention was made with Government support under USDA Agriculture and Food Research Initiative Grant No. 2012-67018-19290 and USDA Multi-state/Hatch Grant No. CONS00916. The government has certain rights in the invention.

### FIELD OF THE INVENTION

The present invention relates to methods of reversing fibrosis or a fibrotic disease, such as fibrosis of the bone marrow, gallbladder, heart, liver, lung, kidney, muscle, pancreas, and/or other soft tissues, in a subject by administering astaxanthin (ASTX) to the subject.

### BACKGROUND OF THE INVENTION

Fibrosis, a disorder characterized by excessive scarring, is thought to be the result of the normal wound healing response gone awry. One hallmark of fibrosis is the excessive production and deposition of collagen and other extracellular matrix components. Causes of fibrosis are diverse, and include trauma, surgery, infection, and exposure to toxins (including environmental pollutants, alcohol and other toxins). Fibrosis is also associated with various disease states such as diabetes, obesity, and non-alcoholic steatohepatitis. Fibrotic disorders can be characterized as acute or chronic, but share the common characteristic of excessive collagen accumulation and an associated loss of function as normal tissue is replaced or displaced by fibrotic tissue. Organs that are commonly affected by fibrosis include liver, kidney, and lung.

Hepatic stellate cells (HSCs) are primarily responsible for extracellular matrix (ECM) production and fibrosis development in the liver. In the normal liver, quiescent HSCs (qHSCs) are present in the space of Disse and contain cytoplasmic lipid droplets mainly consisting of retinyl esters. In response to injury, qHSCs transdifferentiate to myofibroblast-like cells, which highly express α-smooth muscle actin (α-SMA), a myofibroblast marker, and ECM proteins, e.g., procollagen type I α1 (Col1A1). The activated HSCs (aHSCs) are highly proliferative and migrate to the sites of injury. HSC activation may be a reversible process. When stimulants, such as oxidants or fibrogenic cytokines, are removed, aHSCs are either eliminated through apoptosis or revert to an inactive phenotype, i.e., inactivated HSCs (iHSCs), which share common features with qHSCs but differ in their sensitivity to fibrogenic insults.

Astaxanthin (ASTX) is a xanthophyll carotenoid, which gives reddish appearance to aquatic animals such as salmon and shrimp. Studies have demonstrated that ASTX exerts protective effects against oxidative stress, inflammation, type 2 diabetes, and cardiovascular diseases. However, whether ASTX can prevent the development of liver fibrosis has never been addressed.

While Shen et al. (Mediators of Inflammation 2014) teach the protective effect of astaxanthin on liver fibrosis through modulation of TGF-β1 expression and autophagy, Shen fails to teach or suggest that astaxanthin can reverse fibrosis, a critical distinction in treating fibrotic disorders.

### SUMMARY OF THE INVENTION

The present invention is based, at least in part, on the discovery that astaxanthin (ASTX) reverses the activation of hepatic stellate cells (HSCs). Thus, astaxanthin (ASTX) may be used as a natural anti-fibrogenic agent for the reversal of fibrosis and fibrotic diseases, such as fibrosis of the liver.

Accordingly, in one aspect, the present invention provides a method of reversing fibrosis in a subject comprising administering to the subject an effective amount of astaxanthin (ASTX), a pharmaceutically acceptable salt thereof, or a pharmaceutical composition comprising astaxanthin (ASTX) and a pharmaceutically acceptable excipient, thereby reversing fibrosis in the subject

In another aspect, provided herein is a method of reversing a fibrotic disease in a subject comprising administering to the subject an effective amount of astaxanthin (ASTX), a pharmaceutically acceptable salt thereof, or a pharmaceutical composition comprising astaxanthin (ASTX) and a pharmaceutically acceptable excipient, thereby reversing the fibrotic disease in the subject

In one embodiment of the foregoing aspects, the subject is a mammal. In a particular embodiment, the subject is a human.

In one embodiment, the fibrotic disease is an acute condition. In another embodiment, the fibrotic disease is a chronic condition.

In some embodiments, the fibrotic disease is aberrant wound healing, acute interstitial pneumonitis, arthrofibrosis, asthma, atherosclerosis, bone-marrow fibrosis, cardiac fibrosis, chronic kidney disease, cirrhosis of gallbladder, cirrhosis of liver, colloid and hypertrophic scar, Crohn's disease, cryptogenic organizing pneumonia, cystic fibrosis, desquamative interstitial pneumonia, diffuse parenchymal lung disease, Dupuytren's contracture, endomyocardial fibrosis, fibrosis as a result of Graft-Versus-Host Disease (GVHD), glomerulonephritis, idiopathic interstitial fibrosis, interstitial lung disease, interstitial pneumonitis, keloid scar, hypertrophic scar, liver fibrosis, lymphocytic interstitial pneumonia, morphea, multifocal fibrosclerosis, muscle fibrosis, myelofibrosis, nephrogenic systemic fibrosis, nonspecific interstitial pneumonia, organ transplant fibrosis, pancreatic fibrosis, Peyronie's disease, pulmonary fibrosis, renal fibrosis, respiratory bronchiolitis, retroperitoneal fibrosis, scarring after surgery, scleroderma, subepithelial fibrosis, or uterine fibrosis.

In certain embodiments the subject is suffering from fibrosis of the bone marrow, gallbladder, heart, liver, lung, kidney, muscle, pancreas, penis, and/or uterus,

In a particular embodiment, the subject is suffering from fibrosis of the liver or a fibrosis-related liver disease. In certain other embodiments, the subject suffers from at least one of chronic Hepatitis B, Hepatitis C, non-alcoholic steatophepatitis (NASH), alcoholic liver disease, a metabolic liver disease, Wilson's disease, hemochromatosis, or biliary obstruction.

In a particular embodiment, the method further includes identifying the subject as having fibrosis or a fibrotic disease.

In a particular embodiment, provided herein is a method of reversing fibrosis or a fibrotic disease in a subject, wherein astaxanthin (ASTX) reverses fibrosis.

In yet another embodiment, provided herein is a method of reversing fibrosis or a fibrotic disease in a subject, wherein astaxanthin (ASTX) reverses activation of hepatic stellate cells (HSC).

In one embodiment, provided herein is a method of reversing fibrosis or a fibrotic disease in a subject, wherein astaxanthin (ASTX) inhibits TGFβ1 signaling.

In another embodiment, provided herein is a method of reversing fibrosis or a fibrotic disease in a subject, wherein astaxanthin (ASTX) inhibits HDAC9 expression.

In yet another embodiment, provided herein is a method of reversing fibrosis or a fibrotic disease in a subject, wherein astaxanthin (ASTX) inhibits cellular reactive oxygen species (ROS) accumulation.

In still another embodiment, provided herein is a method of reversing fibrosis or a fibrotic disease in a subject, wherein astaxanthin (ASTX) inhibits expression of myocyte enhancer factor 2 (MEF2).

In one embodiment, provided herein is a method of reversing fibrosis or a fibrotic disease in a subject, wherein astaxanthin (ASTX) inhibits basal expression of fibrogenic genes.

In another embodiment, provided herein is a method of reversing fibrosis or a fibrotic disease in a subject, wherein astaxanthin (ASTX) inhibits TGFβ1-induced expression of fibrogenic genes.

In another aspect, the present invention provides a method of inhibiting the activation of quiescent hepatic stellate cells (qHSCs) or inactivated hepatic stellate cells (iHSCs), by contacting hepatic stellate cells with astaxanthin (ASTX), or a pharmaceutically acceptable salt thereof, thereby inhibiting the activation of quiescent or inactivated hepatic stellate cells.

In another aspect, provided herein is a method of reversing the activation of activated hepatic stellate cells (aHSC), by contacting the activated hepatic stellate cells with astaxanthin (ASTX), or a pharmaceutically acceptable salt thereof, thereby reversing the activation of activated hepatic stellate cells. For example, the ASTX may reverse activated hepatic stellate cells to inactive hepatic stellate cells. Alternatively, or in combination, the ASTX may reverse activated hepatic stellate cells to quiescent hepatic stellate cells.

In one embodiment of the foregoing aspects, the hepatic stellate cells, for example, quiescent, activated or inactivated hepatic stellate cells are murine or human primary hepatic stellate cells (HSCs).

In another embodiment, the quiescent, activated or inactivated hepatic stellate cells are incubated for 2-6 days.

In another embodiment, provided herein is a method of inhibiting TGFβ1 signaling in a hepatic stellate cell (HSC) by contacting the cell with astaxanthin (ASTX).

In yet another embodiment, provided herein is a method of inhibiting activation of the Smad3 pathway in hepatic stellate cells (HSC) by contacting the cell with astaxanthin (ASTX).

In one embodiment, provided herein is a method of inhibiting HDAC9 expression by contacting the cell with astaxanthin (ASTX).

In another embodiment, provided herein is a method of inhibiting cellular reactive oxygen species (ROS) accumulation in hepatic stellate cells (HSC) by contacting the cell with astaxanthin (ASTX).

In yet another embodiment, provided herein is a method of inhibiting expression of myocyte enhancer factor 2 (MEF2) in hepatic stellate cells (HSC) by contacting the cell with astaxanthin (ASTX).

In still another embodiment, provided herein is a method of inhibiting basal expression of fibrogenic genes in hepatic stellate cells (HSC) by contacting the cell with astaxanthin (ASTX).

In another embodiment, provided herein is a method of inhibiting TGFβ1-induced expression of fibrogenic genes in hepatic stellate cells (HSC) by contacting the cell with astaxanthin (ASTX).

### BRIEF DESCRIPTION OF THE DRAWINGS

FIG. 1A is a cytotoxicity measurement for LX-2 cells treated with 0-200 μM ASTX for 24 h. FIG. 1B is a bar graph showing ROS production in LX-2 cells pretreated with 0, 5, 10 or 25 μM ASTX for 24 h, and subsequently exposed to 2 ng/mL TGFβ1. FIG. 1C is a bar graph showing ROS production in LX-2 cells pretreated with 0, 5, 10 or 25 μM ASTX for 24 h, and subsequently exposed to 10 μM tBHP.

FIG. 2A is a line graph showing the results of qRT-PCR analysis to measure fibrogenic gene expression in LX-2 cells that were pretreated with or without 25 μM ASTX for 24 h, and subsequently exposed to 2 ng/mL TGFβ1 for 0-24 h. FIG. 2B is a bar graph showing the results of qRT-PCR analysis of LX-2 cells that were pretreated with 0, 5, 10 or 25 μM ASTX for 24 h, then exposed to 2 ng/mL TGFβ1 for 24 h in the presence of the same ASTX concentration.

FIG. 3A is a bar graph showing the results of qRT-PCR analysis of LX-2 cells that were pre-incubated with or without 25 μM ASTX for 24 h, and then stimulated with 2 ng/mL TGFβ1 in the presence or absence of ASTX for an additional 24 h. FIG. 3B shows the results of western blot analysis to measure α-SMA protein levels using β-tubulin as a loading control of LX-2 cells, pre-incubated with or without 25 μM ASTX for 24 h, and then stimulated with 2 ng/mL TGFβ1 in the presence or absence of ASTX for 12 or 24 h. Representative blot images also are shown from 3 repeats. FIG. 3C shows immunostaining of α-SMA protein (upper) and DAPI (lower) for LX-2 cells pre-incubated with or without 25 μM ASTX for 24 h, and subsequently stimulated with 2 ng/mL TGFβ1 with or without ASTX.

FIG. 4 is a bar graph showing the results of qRT-PCR analysis of LX-2 cells that were transfected with Scramble control or Smad3 siRNA, subsequently pretreated with or without 25 μM ASTX for 12 h, and then activated by 2 ng/mL TGFβ1 for 12 h.

FIG. 5A shows western blot analysis for total and phosphorylated Smad3 for LX-2 cells that were pre-incubated with or without 25 μM ASTX for 24 h and subsequently for an additional 3 h with replenishing ASTX in the media. FIG. 5B shows western blot analysis to measure Smad3 protein in the cytoplasm and nucleus for LX-2 cells that were pre-incubated with or without 25 μM ASTX for 24 h and subsequently for an additional 3 h with replenishing ASTX in the media. FIG. 5C a bar graph showing the results of qRT-PCR analysis of LX-2 cells pre-incubated with 25 μM ASTX for 24 h, then stimulated with 2 ng/mL TGFβ1 for 12 h.

FIG. 6A is a bar graph showing the results of qRT-PCR analysis of primary HSCs isolated from C57BL/6J mice and cultured on an untreated plastic dish for 6 days for activation. ASTX at 25 μM was added to cell medium at day 2 or day 4 for additional 2 days. Data are shown as mean±SEM (n=3). Bars sharing a common letter are not significantly different (P<0.05). FIG. 6B shows immunostaining of α-SMA (upper) and DAPI (lower) for primary HSCs cultured for 4 days for activation, with addition of 25 μM ASTX at day 2 for 2 additional days. FIG. 6C shows the results of in-cell Western analysis to measure α-SMA protein levels. Primary HSCs were co-incubated with ASTX from day 0, 2 or 4 till day 6. α-SMA protein is shown in the upper panel and total cell staining for cell number normalization is shown in the middle panel. The bottom panel shows the merging of α-SMA and total cell stain.

FIG. 7A shows cell morphology at day 2, 4 and 6 at 20× magnification for mouse primary HSCs activated for 6 d in the presence of 25 μM ASTX from day 0, day 2 or day 4. FIG. 7B is a bar graph showing the results of qRT-PCR analysis for mouse primary HSCs activated for 6 d in the presence of 25 μM ASTX from day 0, day 2 or day 4 (n=9). Bars with a different letter within the same day are significantly different (P<0.05). FIG. 7C shows a representative blot of α-SMA protein of control and cells treated with ASTX at day 4 for 2 d.

FIG. 8A is a bar graph showing cellular ROS levels (fluorescent unit/total cell stain) at day 6 in controls and ASTX-treated cells from day 0 for 6 d. FIG. 8B is a bar graph showing the results of qRT-PCR analysis of controls or cells treated ASTX for 2 d from day 4 (n=6). FIG. 8C is a bar graph showing the results of qRT-PCR analysis of HSCs from wild-type and Nrf2−/− mice that were activated for 6 d with ASTX from day 0 for qRT-PCR (n=9). FIG. 8D shows that the anti-fibrogenic activity of ASTX is NRF2-independent.

FIG. 9A shows cell morphology at day 8 or 10 at 20× magnification for qHSCs that were activated for 6 d, and then incubated with ASTX for 2 or 4 d. FIG. 9B is a bar graph showing the results of qRT-PCR analysis of controls or aHSCs treated with ASTX for 2 d (n=6; P<0.05). FIG. 9C shows protein expression of α-SMA at day 8 in controls and cells treated with ASTX for 2 d. FIG. 9D is a bar graph showing the results of qRT-PCR analysis of controls and cells treated with ASTX for 2 d. (n=6, P<0.05).

FIG. 10A is a bar graph showing the results of qRT-PCR analysis at day 2 and 6 of activation (n=4, P<0.05). FIG. 10B is a bar graph showing the results of qRT-PCR analysis of HDAC9 mRNA in HSCs treated with ASTX for an indicated day measured at day 2, 4 and 6 (n=9). FIG. 10C shows protein expression of HDAC isoforms at day 2 (qHSCs) and 6 (aHSCs). FIG. 10D shows protein expression at day 6 in controls and cells treated with ASTX at day 4 for 2 d. FIG. 10E is a bar graph showing the results of qRT-PCR analysis for MEF2 isoforms at day 2, at day 6, and at day 6 of HSCs treated with ASTX for 2 d from day 4 (n=6). FIG. 10F is a bar graph showing the results of qRT-PCR analysis for HDAC9 mRNA for aHSCs at day 6, which were treated with ASTX for 2 d. FIG. 10G is a bar graph showing the results of qRT-PCR analysis for MEF2 isoforms in controls and cells treated with ASTX for 2 d (n=6, P<0.05).

FIG. 11A is a bar graph showing the results of qRT-PCR analysis of α-SMA for human primary HSCs pre-incubated with 25 μM ASTX for 24 h and exposed to TGFβ1 (3 ng/ml) for additional 24 h (n=3). Bars with a different letter are significantly different (P<0.05). FIG. 11B is a bar graph showing the results of qRT-PCR analysis of Col1A1 for human primary HSCs pre-incubated with 25 μM ASTX for 24 h and exposed to TGFβ1 (3 ng/ml) for additional 24 h (n=3). Bars with a different letter are significantly different (P<0.05).

FIG. 12A is a bar graph showing the results of qRT-PCR analysis of fibrogenic genes and HDAC9 in PBC and in normal livers (n=9 (normal) or 7 (PBC)). FIG. 12B is a bar graph showing the results of qRT-PCR analysis of MEF2 isoforms in PBC and in normal livers (n=9 (normal) or 7 (PBC)). FIG. 12C is a representative blot for protein in PBC and in normal livers.

FIG. 13A is a bar graph showing the results of qRT-PCR analysis of LX-2 cells pretreated with ASTX for 12 h, and subsequently stimulated by 3 ng/mL TGFβ1 for 12 h for qRT-PCR (n=3). Bars with a different letter are significantly different (P<0.05). FIG. 13B is a bar graph showing the results of qRT-PCR analysis of LX-2 cells transfected with either Scramble control or HDAC9 siRNA for 24 h, and then pretreated with ASTX for 12 h, after which they were stimulated by 3 ng/mL TGFβ1 for 12 h for qRT-PCR analysis (n=3). Bars with a different letter are significantly different (P<0.05).

### DETAILED DESCRIPTION OF THE INVENTION

The present invention is based, at least in part, on the discovery that astaxanthin (ASTX) not only inhibits, but actually reverses the activation of hepatic stellate cells (HSCs). Specifically, the present inventors have identified that ASTX not only inhibits the activation of quiescent HSCs (qHSCs) or inactivated HSCs (iHSCs), but surprisingly reverts activated HSCs (aHSCs) to inactive HSCs (iHSCs). As such, the present invention provides novel methods for reversing fibrosis and fibrotic diseases.

### Methods of Reversing Fibrosis and Fibrotic Diseases

Accordingly, in one aspect, the present invention is directed to a method of reversing fibrosis in a subject by administering an effective amount of ASTX, a pharmaceutically acceptable salt thereof, or a pharmaceutical composition comprising ASTX and a pharmaceutically acceptable excipient, to the subject suffering from fibrosis and/or a fibrotic disease. In a particular embodiment, the fibrotic disease is liver fibrosis, and the ASTX serves, in particular, to reverse the activation of aHSCs (as well as to prevent the activation of further qHSCs and/or iHSCs) so as to reverse the liver fibrosis.

In another aspect, the present invention is directed to a method of reversing a fibrotic disease by administering an effective amount of ASTX, a pharmaceutically acceptable salt thereof, or a pharmaceutical composition comprising ASTX and a pharmaceutically acceptable excipient, to the subject. In a particular embodiment, the fibrotic disease is liver fibrosis, and the ASTX serves, in particular, to reverse aHSCs by reverting the aHSCs to qHSCs or iHSCs so as to reverse the liver fibrosis in the subject.

As used herein, “reverse” or “reversing” refers to partially or totally reducing the extent of the disease and/or symptoms associated therewith. For example, reversing may refer to the reduction in the extent of the fibrosis or fibrotic disease, for example, by the reduction or removal of pre-existing, excess fibrous connective tissue deposited within and/or on an organ or a tissue (e.g., bone marrow, gallbladder, heart, liver, lung, kidney, muscle, pancreas, penis, soft tissue, and/or uterus). Alternatively or in combination, reversing may refer to the progression of fibrosis from a more severe to a less severe state, for example, non-alcoholic steatohepatitis (NASH)-associated fibrosis or alcoholic steatohepatitis (ASH)-associated fibrosis to non-alcoholic steatohepatitis (NASH) or alcoholic steatohepatitis (ASH), respectively, or to non-alcoholic fatty liver disease (NAFLD) or alcoholic fatty liver disease (AFLD), respectively.

Fibrotic diseases effect many tissues within the body as a result of inflammation or damage. Tissues that can be effected by fibrotic diseases include those of the bone marrow, gallbladder, blood vessels, heart, joints, kidney, liver, lung, muscle, pancreas, penis, skin, soft tissue, eye, adrenal glands, thyroids and/or uterus. Exemplary fibrotic conditions include aberrant wound healing, acute interstitial pneumonitis, arthrofibrosis, asthma, atherosclerosis, bone-marrow fibrosis, cardiac fibrosis, chronic kidney disease, cirrhosis of gallbladder, cirrhosis of liver, colloid and hypertrophic scar, Crohn's disease, cryptogenic organizing pneumonia, cystic fibrosis, desquamative interstitial pneumonia, diffuse parenchymal lung disease, Dupuytren's contracture, endomyocardial fibrosis, fibrosis as a result of Graft-Versus-Host Disease (GVHD), glomerulonephritis, idiopathic interstitial fibrosis, interstitial lung disease, interstitial pneumonitis, keloid scar, hypertrophic scar, liver fibrosis, lymphocytic interstitial pneumonia, morphea, multifocal fibrosclerosis, muscle fibrosis, myelofibrosis, nephrogenic systemic fibrosis, nonspecific interstitial pneumonia, organ transplant fibrosis, pancreatic fibrosis, Peyronie's disease, pulmonary fibrosis, renal fibrosis, respiratory bronchiolitis, retroperitoneal fibrosis, scarring after surgery, scleroderma, subepithelial fibrosis, or uterine fibrosis.

In a particular embodiment, the present invention provides methods for reversing liver fibrosis in a subject by administering ASTX, or a pharmaceutically acceptable salt thereof. Liver fibrosis is the excessive accumulation of extracellular matrix proteins including collagen that occurs in most types of chronic liver diseases. In certain embodiments, advanced liver fibrosis results in cirrhosis and liver failure. In one embodiment, provided is a method for reducing the level of fibrosis in a patient. In certain embodiments, liver fibrosis is caused by hepatitis, chemical exposure, bile duct obstruction, autoimmune disease, obstruction of outflow of blood from the liver, heart and blood vessel disturbance, al-antitrypsin deficiency, high blood galactose level, high blood tyrosine level, glycogen storage disease, diabetes, malnutrition, Wilson's Disease or hemochromatosis. In one embodiment, the level of fibrosis is reduced by more than about 90%. In one embodiment, the level of fibrosis is reduced by at least 80%, at least 70%, at least 60%, at least 50%, at least 40%, at least 30%, at least 20%, at least 10%, at least 5% or at least 2%.

In one embodiment, astaxanthin (ASTX) reduces the level of fibrogenesis. Liver fibrogenesis is the process leading to the deposition of an excess of extracellular matrix components in the liver known as fibrosis. Fibrogenesis is observed in a number of conditions such as chronic viral hepatitis B and C, alcoholic liver disease, drug-induced liver disease, hemochromatosis, auto-immune hepatitis, Wilson's disease, primary biliary cirrhosis, sclerosing cholangitis, liver schistosomiasis and others. In one embodiment, the level of fibrogenesis is reduced by more than about 90%. In one embodiment, the level of fibrogenesis is reduced by at least 80%, at least 70%, at least 60%, at least 50%, at least 40%, at least 30%, at least 20%, at least 10%, at least 5% or at least 2%.

In a particular embodiment, the present invention provides methods for reversing a fibrosis-related liver disease in a subject by administering ASTX, or a pharmaceutically acceptable salt thereof. Such fibrosis-related liver disease may be one of non-alcoholic fatty liver disease (NAFLD), alcoholic fatty liver (AFL), non-alcoholic steatohepatitis (NASH), alcoholic steatohepatitis (ASH), non-alcoholic steatohepatitis associated fibrosis, alcoholic steatohepatitis associated fibrosis, non-alcoholic cirrhosis (e.g., primary biliary cirrhosis) and alcoholic cirrhosis. In a particular embodiment, the fibrosis-related liver disease is a non-alcoholic liver disease including, but not limited to, non-alcoholic fatty liver disease (NAFLD), non-alcoholic steatohepatitis (NASH), non-alcoholic steatohepatitis-associated fibrosis, and non-alcoholic cirrhosis. In another embodiment, the fibrosis-related liver disease is an alcoholic liver disease including, but not limited to, alcoholic fatty liver (AFL), alcoholic steatohepatitis (ASH), alcoholic steatohepatitis-associated fibrosis, and alcoholic cirrhosis.

In addition to the primary fatty liver diseases set forth above, the fibrosis-related liver disease may be a secondary fatty acid liver disease associated with, for example, ALD, hepatitis (e.g., viral, alcoholic or chronic hepatitis), total parental nutrition (TPN), steroid treatment, tamoxifen, gastrointestinal operations, Reye's Syndrome, gastrointestinal disorders such as Intestinal Bacterial Overgrowth (IBO), gastroparesis and irritable bowel (IBS) disorders, and chemotherapy.

In certain embodiments, the methods provided herein include the reversal of acute and/or chronic fibrosis-related liver diseases. In one embodiment, the methods are for reversal of an acute fibrosis-related liver disease. In one embodiment, the methods are for reversal of a chronic fibrosis-related liver disease. In one embodiment, the methods are for reducing liver damage associated with chronic and/or acute fibrosis-related liver disease.

In one embodiment, the fibrosis-related liver disease is a disorder that results from an injury to the liver. In one embodiment, injury to the liver is caused by toxins, including alcohol, drugs, impurities in foods, and the abnormal build-up of normal substances in the blood. In another embodiment, injury to the liver is caused by an infection or by an autoimmune disorder. In certain embodiments, the exact cause of the injury is not known.

As used herein, the term “subject” includes a human subject, although it is to be understood that the methods described herein are effective with respect to all vertebrate species, which are intended to be included in the term “subject”. Accordingly, a “subject” can include a human subject for medical purposes, such as for the treatment of an existing condition or disease or the prevention of the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein.

In general, the “effective amount” of ASTX refers to the amount necessary to elicit the desired biological response. As will be appreciated by the skilled artisan, the effective amount of ASTX may vary depending on such factors as the desired biological endpoint, the agent to be delivered, the composition of the encapsulating matrix, the target tissue, and the like.

### Astaxanthin

As used herein, “astaxanthin” (ASTX) refers to a xanthophyll carotenoid, having a chemical structure represented by Formula (I):

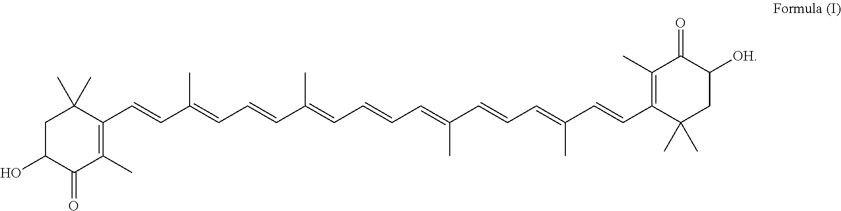 [SMILES: CC1=C(/C=C/C(C)=C/C=C/C(C)=C/C=C/C=C(C)/C=C/C=C(C)/C=C/C2=C(C)C(=O)C(O)CC2(C)C)C(C)(C)CC(O)C1=O]

In the practice of the methods of this invention, astaxanthin from any source, whether natural or synthetic, can be used. Synthetic methods for preparing astaxanthin are known (R. D. G. Cooper et al., J. Chem Soc. Perkins Trans. I, (1975) p. 2195; F. Kienzle, H. Mayer, Helv. Chim. Acta., (1978) Vol. 61, p. 2609) as are methods of isolating astaxanthin from natural sources (Tischer, Z., Physiol. Chem., (1941) Vol. 267 p. 281; Seybold and Goodwin, Nature, (1959) Vol. 184, p. 1714). Haematococcus pluvialis microalgae is a preferred natural, commercially available source of the astaxanthin for use in the methods of this invention. Thus, astaxanthin can be administered in a pure form as synthesized or isolated from natural sources. Alternatively, astaxanthin is administered as part of a composition comprising protein, carbohydrate, and fatty acids. When Haemotococcus pluvialis microalgae is used as the source of astaxanthin, the composition is administered as derived from the microalgae, comprising the natural protein, carbohydrate, and fatty acid components of the microalgae. Such microalgae is commercially available (AstaReal Technologies, Inc. (Fuji Chemical Group), Moses Lake, Wash.) and generally comprises as major components (by weight), from 5% astaxanthin, 10% protein, 40% carbohydrates, 3% ash, 40% fat, and 3% moisture. The composition can further comprise minor components including iron, magnesium, calcium, biotin, L-carnitine, folic acid, niacin, pantothenic acid, and vitamins B1, B2, B6, B12, C, and E.

Thus, in the practice of the invention, the composition can comprise from about 0.1% to about 75% by weight astaxanthin, from about 0.1% to about 99.9% by weight protein, from about 0.1% to about 99.9% by weight carbohydrate, and from about 0.1% to 99.9% fatty acids. In a certain embodiment, the composition comprises from about 15% to about 35% by weight protein, from about 15% to about 60% by weight carbohydrates, from about 1% to about 30% by weight fatty acids, and from about 0.1% to about 4.0% by weight of the carotenoid astaxanthin. In a preferred embodiment, the composition comprises 5% astaxanthin, 10% protein, 40% carbohydrates, 3% ash, 40% fat, and 3% moisture.

Astaxanthin (ASTX) can be administered per se as well as in the form of pharmaceutically acceptable esters, salts, and ethers, as well as other physiologically functional derivatives of such compounds. Astaxanthin (ASTX) can be amorphous or polymorphic. The term “crystal polymorphs”, “polymorphs”, or “crystal forms” means crystal structures in which a compound (or a salt or solvate thereof) can crystallize in different crystal packing arrangements, all of which have the same elemental composition. Different crystal forms usually have different X-ray diffraction patterns, infrared spectral, melting points, density hardness, crystal shape, optical and electrical properties, stability and solubility. Examples of crystal lattice forms include, but are not limited to, cubic, isometric, tetragonal, orthorhombic, hexagonal, trigonal, triclinic, and monoclinic. Recrystallization solvent, rate of crystallization, storage temperature, and other factors may cause one crystal form to dominate. Crystal polymorphs of the compounds can be prepared by crystallization under different conditions.

Additionally, astaxanthin (ASTX), for example, the salts of the compounds, can exist in either hydrated or unhydrated (the anhydrous) form or as solvates with other solvent molecules. “Solvate” means solvent addition forms that contain either stoichiometric or non-stoichiometric amounts of solvent. Some compounds have a tendency to trap a fixed molar ratio of solvent molecules in the crystalline solid state, thus forming a solvate. If the solvent is water, the solvate formed is a hydrate; and if the solvent is alcohol, the solvate formed is an alcoholate. Hydrates are formed by the combination of one or more molecules of water with one molecule of the substance in which the water retains its molecular state as H₂O.

Non-limiting examples of hydrates include monohydrates, dihydrates, etc. Non-limiting examples of solvates include ethanol solvates, acetone solvates, etc.

Examples of pharmaceutically acceptable acid addition salts include those formed with inorganic acids such as hydrochloric acid, hydrobromic acid, sulfuric acid, nitric acid, phosphoric acid, and the like; as well as organic acids such as acetic acid, trifluoroacetic acid, propionic acid, hexanoic acid, cyclopentanepropionic acid, glycolic acid, pyruvic acid, lactic acid, oxalic acid, maleic acid, malonic acid, succinic acid, fumaric acid, tartaric acid, citric acid, benzoic acid, cinnamic acid, 3-(4-hydroxybenzoyl)benzoic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, p-toluenesulfonic acid, and salicylic acid and the like.

Examples of a pharmaceutically acceptable base addition salts include those formed when an acidic proton present in the parent compound is replaced by a metal ion, such as sodium, potassium, lithium, ammonium, calcium, magnesium, iron, zinc, copper, manganese, aluminum salts and the like. Preferable salts are the ammonium, potassium, sodium, calcium, and magnesium salts. Salts derived from pharmaceutically acceptable organic non-toxic bases include, but are not limited to, salts of primary, secondary, and tertiary amines, substituted amines including naturally occurring substituted amines, cyclic amines and basic ion exchange resins. Examples of organic bases include isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, tromethamine, N-methylglucamine, polyamine resins, and the like.

Exemplary organic bases are isopropylamine, diethylamine, ethanolamine, trimethylamine, dicyclohexylamine, choline, and caffeine.

### Pharmaceutical Compositions Comprising Astaxanthin (ASTX)

In various aspects of the invention, ASTX will be in the form of a pharmaceutical composition containing a pharmaceutically acceptable carrier. As used herein “pharmaceutically acceptable carrier” includes any and all solvents, dispersion media, coatings, antibacterial and antifungal agents, isotonic and absorption delaying agents, and the like that are physiologically compatible. In one embodiment, the pharmaceutically acceptable carrier is not phosphate buffered saline (PBS). In one embodiment, the carrier is suitable for intraocular, topical, parenteral, intravenous, intraperitoneal, or intramuscular administration. In another embodiment, the carrier is suitable for oral administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the ASTX, use thereof in the pharmaceutical compositions of the invention is contemplated. Supplementary active compounds can also be incorporated into the compositions.

Oral Compositions

In the practice of the methods of the invention, the composition may be administered orally in any of the usual solid forms such as pills, tablets, capsules or powders, including sustained release preparations. The term unit dosage form as used in this specification and the claims refer to physically discrete units to be administered in single or multiple dosage to humans, each unit containing a predetermined quantity of active material, i.e., astaxanthin. The quantity of ASTX is calculated to produce the desired therapeutic effect upon administration of one or more of such units. It is understood that the exact treatment level will depend upon the case history of the human subject to be treated. The precise treatment level can be determined by one of ordinary skill in the art without undue experimentation, taking into consideration such factors as age, size, severity of condition, and anticipated duration of administration of compounds, among other factors known to those of ordinary skill.

Unit dosages can range from about 0.02 mg to about 100 mg of astaxanthin, preferably from about 0.2 mg to about 50 mg of astaxanthin, most preferably about 1 mg to about 36 mg of astaxanthin. The doses can be administered in any convenient dosing schedule to achieve the stated beneficial effects. For example, the doses can be taken 1, 2, 3, 4, 5 or more times daily. Preferably 3 doses are taken daily. Most preferably, the doses are taken at meal times. The dosages may be taken orally in any suitable unit dosage form such as pills, tablets, and capsules.

Exemplary carriers for oral compositions include a solid or liquid filler, diluent, or encapsulating substance. Some examples of the substances that can act as carriers are sugars such as lactose, glucose, and sucrose; starches such as corn starch and potato starch; cellulose and its derivatives, such as sodium carboxymethylcellulose, ethylcellulose, cellulose acetate; powdered tragacanth; malt; gelatin; talc; stearic acid; magnesium stearate; calcium sulfate; vegetable oils, such as peanut oil, cottonseed oil, sesame oil, olive oil, corn oil and of the broma; polyols such as propylene glycol, glcerin, sorbitol, mannitol, and polyethylene glycol; agar, alginic acid; pyrogen-free water; isotonic saline; ethyl alcohol and phosphate buffer solutions, as well as other non-toxic compatible substances used in preparation of formulations. Wetting agents and lubricants such as sodium lauryl sulfate, as well as coloring agents, flavoring agents, and preservatives can also be present. Dye stuffs or pigments may be added to the tablets, for example, for identification or in order to characterize combinations of active doses.

Other preparations that can be used orally include push-fit capsules made of gelatin, as well as soft, sealed capsules made of gelatin and a plasticizer such as glycerol or sorbitol. The push-fit capsules can contain the active compounds in the form of granules, which may be mixed with fillers such as lactose, binders such as starches, and/or lubricants such as talc or magnesium stearate and, optionally, stabilizers. In soft capsules, the ASTX is preferably dissolved or suspended in suitable liquids, such as fatty oils, or liquid paraffin. In addition, stabilizers may be added.

Powders are prepared by comminuting the compositions of the present invention to a suitable fine size and mixing with a similarly comminuted diluent pharmaceutical carrier such as an edible carbohydrate material as, for example, starch. Sweetening, flavoring, preservative, dispersing and coloring agents can also be present.

Capsules are made by preparing a powder mixture as described above and filling formed gelatin sheaths. A lubricant such as talc, magnesium stearate and calcium stearate can be added to the powder mixture as an adjuvant before the filling operation; a glidant such as colloidal silica can be added to improve flow properties; a disintegrating or solubilizing agent may be added to improve the availability of the medicament when the capsule is ingested.

Tablets are made by preparing a powder mixture, granulating or slugging, adding a lubricant and disintegrant and pressing into tablets. A powder mixture is prepared by mixing the compositions of the present invention, suitable comminuted, with a diluent or base such as starch, sucrose, kaolin, dicalcium phosphate, and the like. The powder mixture can be granulated by wetting with a binder such as syrup, starch paste, acacia mucilage or solutions of cellulosic or polymeric materials and forcing through a screen. As an alternative to granulating, the powder mixture can be run through the tablet machine and the resulting imperfectly formed slugs broken into granules. The granules can be lubricated to prevent sticking to the tablet forming dies by means of the addition of stearic acid, a stearate salt, talc or mineral oil. The lubricated mixture is then compressed into tablets. The ASTX can also be combined with free flowing inert carriers and compressed into tablets directly without going through the granulating or slugging steps. A protective coating consisting of a sealing coat of shellac, a coating of sugar or polymeric material and a polish coating of wax can be provided. Dye stuffs or pigments may be added to the tablets, for example, for identification or in order to characterize combinations of active doses. In tablet form the carrier comprises from about 0.1% to 99.9% by weight of the total composition.

Oral compositions generally include an inert diluent or an edible carrier. They can be enclosed in gelatin capsules or compressed into tablets. For the purpose of oral therapeutic administration, ASTX can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic, acid, Primogel, or corn starch; a lubricant such as magnesium stearate or Sterotes; a glidant: such as colloidal silicon dioxide; a sweetening agent such as sucrose or saccharin; or a flavoring agent such as peppermint, methyl salicylate, or orange flavoring.

In one embodiment, the compositions of the invention are prepared with carriers that will protect ASTX against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These may be prepared according to methods known to those skilled in the art, for example, as described in U.S. Pat. No. 4,522,811.

Other Modes of Administration

In another embodiment, the pharmaceutical compositions of the present invention would be administered in the form of injectable compositions. The compositions can be prepared as an injectable, either as liquid solutions or suspensions. The preparation may also be emulsified. Suitable excipients are, for example, water, saline, dextrose, glycerol, ethanol, or the like and combinations thereof. In addition, if desired, the preparation may contain minor amounts of auxiliary substances such as wetting or emulsifying agents, pH-buffering agents, adjuvants, or immunopotentiators.

Sterile injectable solutions can be prepared by incorporating the compositions of the invention in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Nasal compositions generally include nasal sprays and inhalants. Nasal sprays and inhalants can contain one or more active components and excipients such as preservatives, viscosity modifiers, emulsifiers, buffering agents and the like. Nasal sprays may be applied to the nasal cavity for local and/or systemic use. Nasal sprays may be dispensed by a non-pressurized dispenser suitable for delivery of a metered dose of the active component. Nasal inhalants are intended for delivery to the lungs by oral inhalation for local and/or systemic use. Nasal inhalants may be dispensed by a closed container system for delivery of a metered dose of one or more active components.

In one embodiment, nasal inhalants are used with an aerosol. This is accomplished by preparing an aqueous aerosol, liposomal preparation or solid particles containing the compound. A non-aqueous (e.g., fluorocarbon propellant) suspension could be used. Sonic nebulizers may be used to minimize exposing the agent to shear, which can result in degradation of the compound.

Ordinarily, an aqueous aerosol is made by formulating an aqueous solution or suspension of the agent together with conventional pharmaceutically acceptable carriers and stabilizers. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the ASTX IS formulated into ointments, salves, gels, or creams as generally known in the art.

The compositions of the invention can also be prepared in the form of suppositories (e.g., with conventional suppository bases such as cocoa butter and other glycerides) or retention enemas for rectal delivery.

Dosing

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of nucleic acid molecules described herein can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

Data obtained from cell culture assays and/or animal studies can be used in formulating a range of dosage for use in humans. The dosage typically will lie within a range of circulating concentrations that include the ED₅₀ with little or no toxicity. The dosage may vary within this range depending upon the dosage form employed and the route of administration utilized. The therapeutically effective dose of ASTX can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

In general, pharmaceutical compositions comprising astaxanthin (ASTX) for use in the methods of the invention can be administered in therapeutically effective amounts via any of the usual and acceptable modes known in the art, as described above, either singly or in combination with one or more therapeutic agents. A therapeutically effective amount can vary widely depending on the severity of the disease, the age and relative health of the subject, the potency of the compound used and other factors involved, as readily determinable within the skill of the art. Suitable therapeutic doses of astaxanthin (ASTX) may be in the range of 1 milligram (mg) to 100 milligrams (mg) per recipient per day, and any increment in between, such as, e.g., 1, 2, 3, 5, 10, 25, 50, 75 or 100 mg. In some embodiments, the therapeutic doses of astaxanthin (ASTX) are in the range of 1 mg to 100 mg per recipient per day. In a preferred embodiment, the therapeutic dose of astaxanthin (ASTX) is in the range of 1 mg to 50 mg per recipient per day. Alternatively, the therapeutic dose of astaxanthin (ASTX) is in the range of 6 mg to 36 mg per recipient per day.

A desired dose can be presented as two, three, four, five, six, or more sub-doses administered at appropriate intervals throughout the day. These sub-doses can be administered in unit dosage forms, for example, containing from 1 mg to 100 mg (e.g. about 1, 2, 3, 5, 10, 15, 18, 20, 25, 30, 35, 40, 45, 50, 75 or 100 mg) of ASTX per unit dosage form. Alternatively, if the condition of the recipient so requires, the doses may be administered as a continuous infusion. The mode of administration and dosage forms will affect the therapeutic amounts of the compounds which are desirable and efficacious for the given treatment application.

It is understood, however, that a specific dose level for any particular subject will depend upon a variety of factors including the activity of astaxanthin (ASTX), the age, body weight, general health, sex, diet, time of administration, rate of excretion, drug combination, and the severity of the particular disease being treated and form of administration.

These considerations, as well as effective formulations and administration procedures are well known in the art and are described in standard textbooks.

### GENERAL DEFINITIONS

Although specific terms are employed herein, they are used in a generic and descriptive sense only and not for purposes of limitation. Unless otherwise defined, all technical and scientific terms used herein have the same meaning as commonly understood by one of ordinary skill in the art to which this presently described subject matter belongs.

Following long-standing patent law convention, the terms “a”, “an”, and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise”, “comprises”, and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, parameters, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about”, when referring to a value can be meant to encompass variations of, in some embodiments ±100%, in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

This invention is further illustrated by the following examples which should not be construed as limiting. The contents of all references, patents and published patent applications cited throughout this application, as well as the Figures, are hereby incorporated by reference.

### EXEMPLIFICATION

### Example 1

### Astaxanthin Prevents TGFB1-Induced Pro-Fibrogenic Gene Expression by Inhibiting SMAD3 Activation in Hepatic Stellate Cells

### Materials and Methods

### Isolation of Primary Mouse HSCs

Primary HSCs were isolated from C57BL/6J mouse liver using the collagenase/pronase digestion method (see Weiskirchen et al. Fibrosis Research, 2005, vol. 117, pp. 99-113). In brief, mice were anesthetized with ketamine/xylazine (120/6 mpk) (Butler Schein, Dublin, Ohio). Abdominal and chest cavities were opened to expose the portal vein and to locate the heart and vena cava, respectively. Subsequently, a 20 G×1″ I.V. catheter (Terumo, Somerset, N.J.) was inserted into the vena cava through the right atrium and the portal vein was cut. A mouse liver was perfused at 5 mL/min with 25 mL SC-1 solution consisting of the following (per L); 8000 mg NaCl, 400 mg KCl, 88.17 mg NaH₂PO₄ 2H₂O, 120.45 mg Na₂HPO₄ 12H₂O, 2380 mg 4-(2-hydroxyethyl)-1-piperazineethanesulfonic acid (HEPES), 350 mg NaHCO₃, 190 mg ethylene glycol tetraacetic acid, and 900 mg glucose, pH 7.2-7.3. The liver was, then, digested with 300 μg/mL pronase E (Roche, Mannheim, Germany) in SC-2 solution consisting of 8000 mg NaCl, 400 mg KCl, 88.17 mg NaH₂PO₄ 2H₂O, 120.45 mg Na₂HPO₄ 12H₂O, 2380 mg HEPES, 350 mg NaHCO₃, and 560 mg CaCl₂ 2H₂O, per L, pH 7.2-7.3, followed by 600 μg/mL collagenase D (Roche, Mannheim, Germany) in SC-2 solution at 5 mL/min for 5 min each. The digested liver was harvested and filtered through a 100 μm cell strainer (Fisher Scientific, Pittsburgh, Pa.). The hepatic cells were then centrifuged at 60×g for 1 min to separate non-parenchymal cells from hepatocytes. The supernatant containing non-parenchymal cells was separated based on density using a top layer of Gey's Balanced Salt Solution (GBSS), a middle layer of 10% Nycodenz (Axis-Shield, Scotland) and a bottom layer of 14.5% Nycodenz diluted in GBSS. The tube was centrifuged at 2100×g or 20 min and the primary HSCs were collected from the interface between the GBSS and 10% Nycodenz.

### HSC Culture and Treatment

LX-2 cells were kindly provided by Dr. Scott Friedman at the Icahn School of Medicine at Mount Sinai (New York, N.Y.). Cells were maintained in low-glucose DMEM containing 2% FBS, 4 mM 1-glutamine, 100 U/mL penicillin and 100 μg/mL streptomycin in a 37° C. humidified cell culture chamber providing 5% CO₂. LX-2 cells were incubated with varying concentrations of ASTX for 12 or 24 h, and subsequently activated by 2 ng/mL of TGFβ1 (Peprotech, Rocky Hill, N.J.) for 1 to 24 h. Primary mouse HSCs were plated on untreated petri dishes (BD Falcon, Franklin Lakes, N.J.) and maintained in low-glucose DMEM supplemented with 10% FBS, 4 mM L-glutamine, 100 U/mL penicillin and 100 μg/mL streptomycin in a 37° C. humidified cell culture chamber under 5% CO₂. ASTX (25 μM) was added at day 2 or day 4 after plating until day 6 with daily media change. All cell culture supplies were purchased from HyClone (Thermo Scientific, Logan, Utah).

ASTX was kindly provided by Fuji Chemical Industry Co., Ltd. (Toyama, Japan). ASTX stock solution (10 mM) was prepared in DMSO and stored at −80° C. until use. Before cell treatments, ASTX stock was incubated in 70° C. for 10 min, after which the ASTX stock was dissolved in FBS and then diluted in cell culture medium to obtained desired concentrations. The final FBS concentration in ASTX-containing medium was 2% and therefore the same amount FBS and DMSO was added to controls. All ASTX-containing solutions were kept in the dark to prevent any light-induced degradation.

### Cytotoxicity Test

LX-2 cells were incubated with 0-200 μM ASTX for 24 h and its cytotoxicity was measured using a Cell Counting Kit-8 (Dojindo Inc., Rockville, Md.) as previously described (see Yang et al. Food Chem. Toxicol. 2011, vol. 49, pp. 1560-1564). Positive control (0.5 mM SDS) was run in parallel.

### Reactive Oxygen Species (ROS) Measurement

Cellular ROS levels were measured in LX-2 cells as previously described (see Lee et al. J. Nutr. Biochem. 2013, vol. 25, iss. 4, pp. 404-411). Briefly, LX-2 cells were plated in a black 24-well plate (Wallac Oy, Turku, Finland). When cells reached ˜90% confluency, they were pre-incubated with 5, 10 or 25 μM ASTX for 24 h and subsequently stimulated with 2 ng/mL TGFβ1 or 10 μM tert-butyl hydrogen peroxide (tBHP, Sigma, St. Louis, Mo.) for additional 24 h. cells were then incubated with 5 μM dichlorofluorescein (Sigma, St. Louis, Mo.) for 30 min and fluorescence was read at an excitation wavelength of 485 nm and an emission wavelength of 530 nm. The data were expressed as fluorescent intensity per μg of cell protein.

Quantitative Real-Time PCR (qRT-PCR)

Total RNA extraction, cDNA synthesis, and qRT-PCR were conducted using a Bio-Rad CFX96 Real-Time system (Bio-Rad, Hercules, Calif.) as previously described (see Park et al. Nutr. Res. 2008, vol. 28, pp. 83-91; Rasmussen et al. J. Nutr. 2008, vol. 138, pp. 476-481). Gene sequences were obtained from the GenBank database and primers were designed using Beacon Designer (Premier Biosoft, Palo Alto, Calif.). Primer sequences are available (see supplemental table of Yang et al. Biochimica et Biophysica Acta 2015, vol. 1850, pp. 178-185).

### Western Blot Analysis

Whole cell lysates were prepared to conduct Western blot as we previously described (Id.) using antibodies against α-SMA (Sigma, St. Louis, Mo.), Smad3 (Millipore, Billerica, Mass.), and phospho-Smad3 (Cell Signaling, Danvers, Mass.). β-Tubulin (Santa Cruz Biotechnology, Santa Cruz, Calif.) or β-actin (Sigma, St. Louis, Mo.) was used as a loading control. For Western blot of nuclear and cytoplasmic cell fractions, the fractions were prepared using a nuclear extraction kit (Cayman, Ann Arbor, Mich.) as previously described (see Ku et al. Biochim. Biophys. Acta Gen. Subj. 2013, vol. 1830, pp. 2981-2988). Glyceraldehyde 3-phosphate dehydrogenase (GAPDH) (Santa Cruz Biotechnology, Santa Cruz, Calif.) and lamin B (Santa Cruz Biotechnology) were used as a loading control for cytoplasmic and nuclear fractions, respectively. Blots were developed using Westpico horseradish peroxidase chemiluminescence (Pierce, Rockford, Ill.) and images were analyzed using a Chemidoc XRS+system (Bio-Rad) and Image Lab software (Bio-Rad).

### Immunocytochemistry

LX-2 cells and primary HSCs were plated on a Millicell E Z slide (Millipore, Billerica, Mass.). After treatment with ASTX, cells were fixed with 4% formaldehyde for 10 min and subsequently blocked with 1% BSA in 1×TBS containing 0.1% Triton X-100 (TBS-T) for 30 min at room temperature. Fixed cells were then incubated with α-SMA antibody overnight at 4° C., after which they were incubated with anti-mouse DyLight 488 conjugated secondary antibody (Abcam, Cambridge, Mass.) in the dark for 1 h at room temperature. Cell nuclei were visualized by DAPI staining (100 ng/mL in PBS) for 10 min. After washing the cells with TBS-T, Prolong Gold anti-fade reagent (Invitrogen, Grand Island, N.Y.) was added onto the cells and images were taken with an AxioCam MRc camera (Carl Zeiss Microscopy, Jena, Germany).

### In-Cell Western

Primary HSCs were incubated on an uncoated μ-Plate (Ibidi, Verona, Wis.). After 6 days of activation with or without ASTX, HSCs were fixed in 4% formaldehyde for 20 min at room temperature and washed with 1% TBS-T for 5 times for 5 min each. The cells were blocked with Odyssey® Blocking Buffer for 30 min, then incubated with α-SMA primary antibody for 2 h at room temperature. Subsequently, the cells were co-incubated with IRDye 800CW secondary antibody and CellTag 700 stain, a fluorescent stain for cell number normalization, in the dark for 1 h at room temperature. Pictures were taken using an Odyssey CLx Imager (Li—COR, Lincoln, Nebr.) and signals were quantified by a Li—COR Image Studio software with normalization using a signal of CellTag 700 stain. All reagents and imaging tools for In-cell Western were purchased from Li—COR.

Small Interference RNA (siRNA) Transfection

LX-2 cells were transfected with either Silencer® Negative Control scrambled siRNA (Ambion, Invitrogen, Grand Island, N.Y.) or siGENOME SMARTpool SMAD3 siRNA (siGenome, Thermo Scientific, Logan, Utah) as described previously (see Angulo N. Engl. J. Med. 2002, vol. 346, pp. 1221-1231). Twenty four hours after the transfection, cells were pretreated with 25 μM ASTX for 12 h, after which they were stimulated by 2 ng/mL TGFβ1 for additional 12 h for subsequent qRT-PCR analysis.

### Statistical Analysis

One-way analysis of variance (ANOVA) and the Newman Keul pairwise post-hoc test were used to detect significant differences between groups. Statistical analyses were conducted by using GraphPad Prism6 (GraphPad Software, La Jolla, Calif.). P values less than 0.05 were considered significant and all values were presented as mean±SEM.

### Results

ASTX Prevented TGFβ1 and tBHP-Induced ROS Accumulation in LX-2 Cells

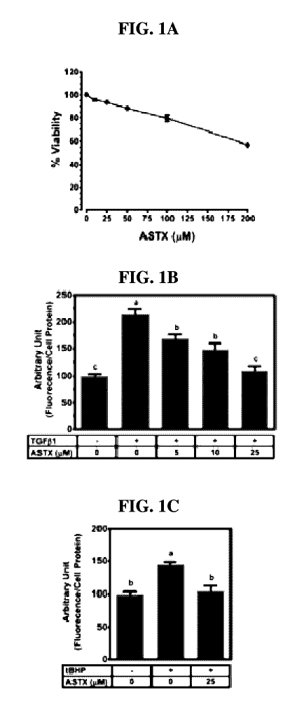

ASTX showed minimal cytotoxicity in LX-2 cells with more than 90% cell viability up to 25 μM (FIG. 1A).

Excessive ROS accumulation has been shown to activate HSCs, which is a key event in the development of hepatic fibrosis. As ASTX is a potent antioxidant, it was determined whether ASTX attenuates ROS accumulation induced by TGFβ1 or tBHP in LX-2 cells. Both TGFβ1 and tBHP significantly increased cellular ROS levels, which were decreased by ASTX (FIGS. 1B and 1C). At 25 μM, ASTX completely abolished the increase in cellular ROS levels induced by TGFβ1 and tBHP.

### ASTX Attenuated the TGFβ1-Induced Expression of Fibrogenic Genes in LX-2 Cells

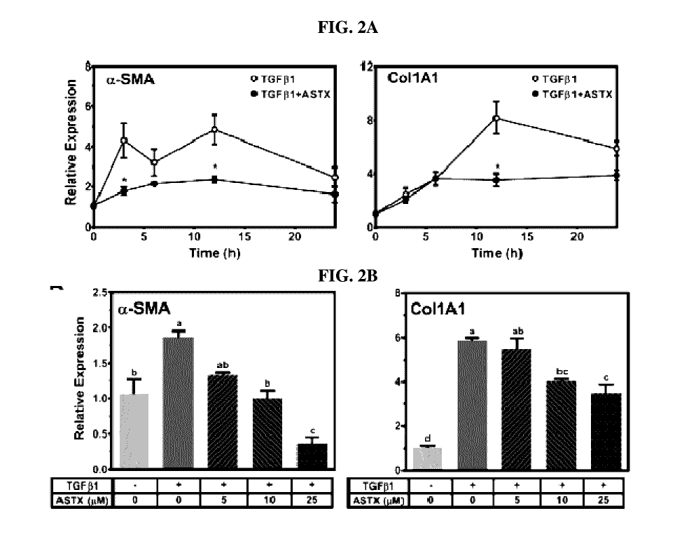

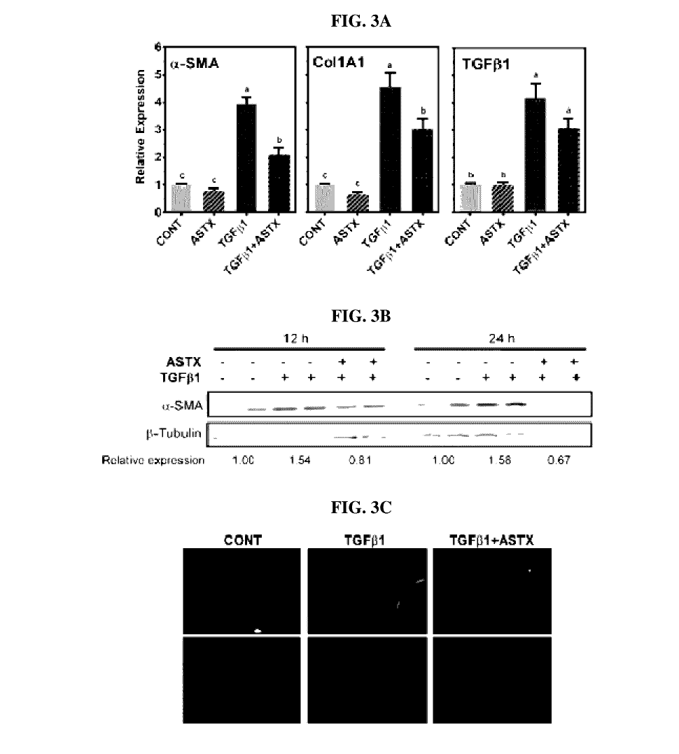

TGFβ1 is the most potent pro-fibrogenic cytokine in HSCs and therefore it was necessary to determine the effect of ASTX on TGFβ1-induced fibrogenic response in LX-2 cells. TGFβ1 significantly increased mRNA abundance of α-SMA and Col1A1 as early as 3 h and the mRNA levels reached the highest at 12 h (FIG. 2A). ASTX significantly inhibited the induction of α-SMA at 3 and 12 h and Col1A1 at 12 h of TGFβ1 stimulation. Induction of the fibrogenic genes by TGFβ1 was significantly inhibited by 10 and 25 μM of ASTX (FIG. 2B). Based on this finding, ASTX treatment for 12 h at 25 μM was chosen for the rest of the study. ASTX did not alter the basal expression levels of α-SMA and Col1A1 mRNA in LX-2 cells, but significantly attenuated TGFβ1-induced expression of these genes (FIG. 3A). ASTX also significantly decreased the protein levels of α-SMA that were induced by TGFβ1 (FIGS. 3B and 3C).

### Smad3 Plays a Major Role in the Induction of Fibrogenic Genes by TGFβ1 in LX-2 Cells

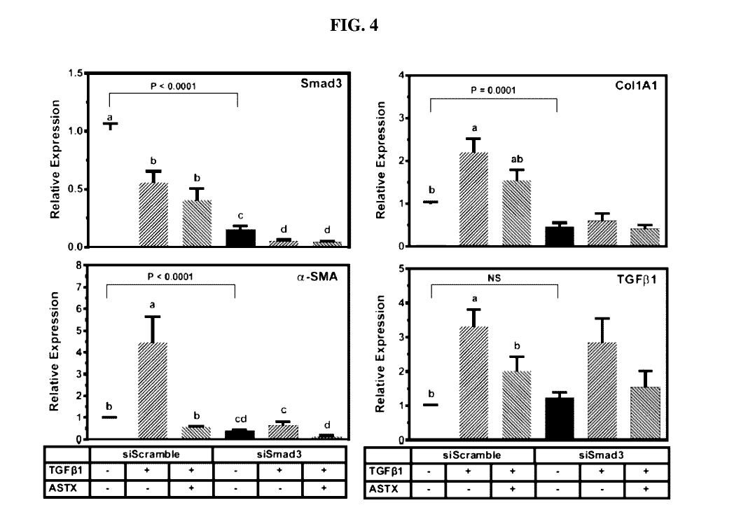

As Smad3 is known to mediate TGFβ1 signaling for fibrogenic responses, the involvement of Smad3 in the repressive effect of ASTX on the induction of fibrogenic genes by TGFβ1 was evaluated. In the LX-2 cells transfected with scrambled control, the induction of fibrogenic genes by TGFβ1 was attenuated by ASTX (FIG. 4). When Smad3 was knocked down by ˜80%, the basal mRNA levels of α-SMA and Col1A1, but not TGFβ1, were significantly decreased compared with scrambled control. TGFβ1 did not induce the expression of α-SMA and Col1A1 but its own mRNA levels were not altered when Smad3 was deficient.

### ASTX Attenuates Phosphorylation and Nuclear Translocation of Smad3 in LX-2 Cells

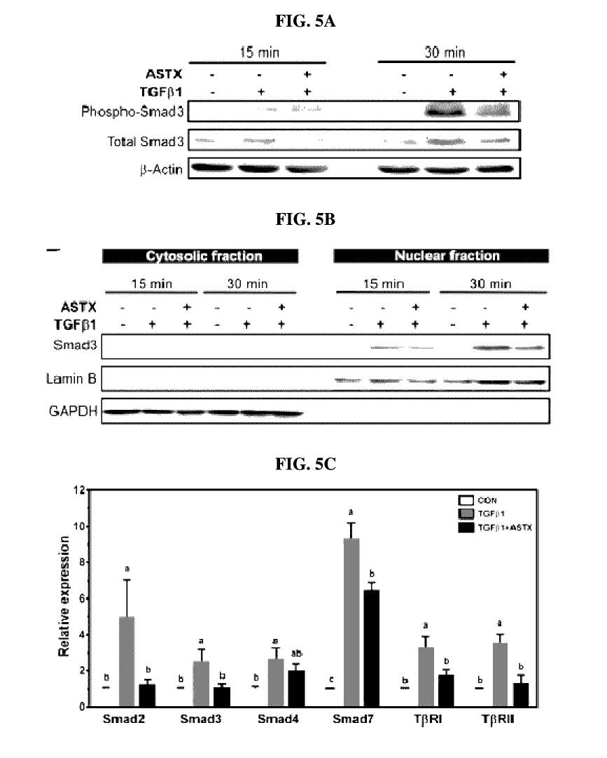

As the anti-fibrogenic effect of ASTX is likely mediated via Smad3, whether ASTX can inhibit Smad3 activation, i.e., phosphorylation and nuclear translocation, was evaluated in LX-2 cells. TGFβ1 stimulation markedly increased the levels of phosphorylated Smad3 at 30 min and ASTX treatment significantly inhibited the TGFβ1-induced phosphorylation (FIG. 5A). Smad3 phosphorylation is an important determinant of its nuclear translocation. Consistent with the reduction in phosphorylated Smad3 by ASTX, the nuclear translocation of Smad3 was also reduced by ASTX, whereas TGFβ1 treatment increased nuclear Smad3 levels (FIG. 5B). The activation of Smad3 requires other players in transducing TGFβ1 signaling. Upon TGFβ1 binding to TβRII, the receptor activates TβRI, which in turn phosphorylates Smad2 and Smad3. The phosphorylation of Smad2 and Smad3 allows them to complex with Smad4 for nuclear translocation. Smad7, transcriptionally induced by Smad3, is known to interact with TβRI through its carboxyl-terminal Mad homology 2 domains and therefore competes with Smad3 for binding to TβRI. It was found that the mRNA levels of Smad2, Smad3, Smad4, and Smad7 were significantly increased by TGFβ1 (FIG. 5C). However, the increase was abolished or significantly attenuated by ASTX except Smad4. Furthermore, ASTX almost completely abolished the TGFβ-induced increases in mRNA levels of both TβRI and TβRII.

### ASTX Prevents α-SMA Elevation During Activation of Mouse Primary HSCs

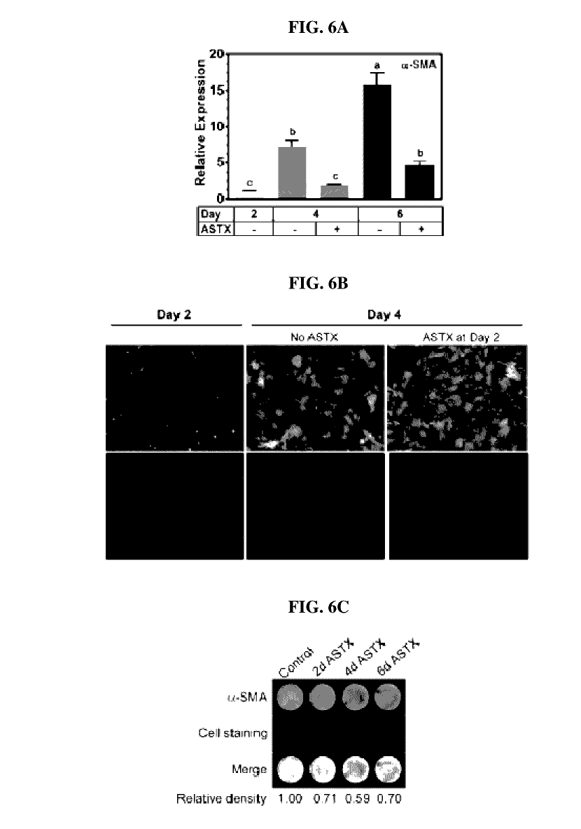

To gain insight into the role of ASTX in the early activation of quiescent HSCs, primary HSCs isolated from C57BL/6J mice were cultured on untreated petri dishes for 4 or 6 days for activation, with the addition of ASTX at day 2 or day 4, respectively. In the absence of ASTX, the expression of α-SMA, a prominent marker for the activation of quiescent HSCs, was significantly increased with time. The addition of ASTX for 2 days during the HSC activation significantly reduced α-SMA mRNA levels (FIG. 6A). At day 4 of incubation, the protein levels of α-SMA were increased, whereas the addition of ASTX for 2 days markedly decreased the protein levels (FIG. 6B). Compared with control cells cultured without ASTX for 6 days, ASTX treatment at days 0, 2, and 4 decreased α-SMA protein by 30-40% (FIG. 6C).

### Discussion

Under the condition of chronic liver injury, sustained activation of HSCs results in excessive deposition of extracellular matrix, leading to the development of fibrosis. Therefore, identification of anti-fibrogenic agents that can inhibit HSC activation may effectively lower the risk of hepatic fibrosis. In the present study, we demonstrated that ASTX represses the expression of TGFβ1-induced fibrogenic genes by inhibiting Smad3 activation in HSCs.

In the foregoing Example 1, ASTX decreased cellular ROS levels induced by TGFβ1 or tBHP (see Example 1.21). Without being bound by any one particular theory, the ROS reduction by ASTX may be achieved by scavenging radicals as it has been shown to scavenge peroxyl and hydroxyl radicals and/or by interfering with signaling pathways that can lead to ROS production. It has been shown that TGFβ1 up-regulates NADPH oxidase 4 (NOX4), a key enzyme for ROS production in HSCs, and inhibition of TβRI activity reduces NOX4 expression and ROS production. Therefore, the afore-mentioned observations that ASTX decreased the expression of TβRI and other TGFβ signaling intermediates suggests that ASTX inhibits the upstream signal for NOX4 transcription, preventing ROS generation. In fact, ASTX decreased TGFβ1-induced NOX4 mRNA levels although it did not reach statistical significance (P=0.074).

TGFβ1 is known to be the most potent pro-fibrogenic cytokine. As shown above, ASTX inhibits the TGFβ1-induced expression of fibrogenic genes, such as α-SMA and Col1A1. The expression of α-SMA is primarily regulated by Smad3, whereas Col1A1 is known to be regulated by Smad3 and specificity protein 1 (Sp1). Further, as shown above, Smad3 knockdown by ˜80% significantly decreased the basal expression of α-SMA and Col1A1, and TGFβ1 did not induce both gene expression when Smad3 was deficient. The data suggest that Smad3 plays a critical role in transducing TGFβ1 signaling for the induction of fibrogenic genes in HSCs. Although ASTX inhibited the TGFβ1-induced Col1A1 and α-SMA expression in the presence of functional Smad3, it had a minimal inhibitory effect on the expression of α-SMA in Smad3-deficient cells. This observation strengthens the notion that the inhibitory effect of ASTX on α-SMA and Col1A1 expression is likely mediated via Smad3. Other transcription factors, such as Krüppel-like factors (KLF) and CCAAT/enhancer binding protein β (CEBPβ), have also been shown to be involved in TGFβ1-induced fibrogenic gene expression. However, the failure to induce α-SMA and Col1A1 expression by TGFβ1 when Smad3 is deficient strongly suggests that Smad3, but not KLF and CEBPβ, is the most critical transcription factor for the induction of the fibrogenic genes in response to TGFβ1 signaling in HSCs. ASTX also inhibited an increase in TGFβ1 mRNA by itself regardless of Smad3. Transcription of TGFβ1 has been suggested to be regulated mainly by Sp1 and KLF6. Therefore, without being bound by any one particular theory, ASTX may have a repressive effect on the activity of other TGFβ1-sensitive transcription factors in addition to Smad3.

Smad3 is one of the downstream effectors of TGFβ1 signaling that induces fibrogenesis in HSCs. Binding of TGFβ1 to TβRII, a cell surface TGFβ1 receptor, phosphorylates TβRI, which subsequently phosphorylates Smad2 and Smad3 for the induction of fibrogenic response. In particular, Smad3 has been shown to be indispensable for TGFβ-induced fibrogenic gene expression. Phosphorylation of Smad2 and Smad3 is required to make a complex with Smad4, which translocates to the nucleus for transcriptional induction. As shown above, in LX-2 cells, TGFβ1 was shown to induce the expression of Smad2, 3, and 7, TβRI and TβRII, which was attenuated by ASTX. The inhibitory action of ASTX in the expression of these intermediate effectors in TGFβ1 signaling may explain how ASTX prevents Smad3 activation, consequently inhibiting its target gene expression. Sp1 has been shown to transcriptionally regulate the expression of fibrogenic genes such as TGFβ1, Col1A1, TβRI, and TβRII. The down-regulation of TGFβ1, TβRI and TβRII by ASTX in TGFβ1-stimulated LX-2 cells, as observed here, suggests that ASTX also inhibits Sp1.

Primary mouse HSCs were also used to gain insight into the effect of ASTX on the early stage of HSC activation. Primary quiescent HSCs are activated when cultured on an untreated plastic dish up to 7 days. During the activation, quiescent HSCs are transdifferentiated into highly proliferative, myoblast-like cells that are characterized by the loss of lipid droplets and high α-SMA expression, an activation marker. As shown above, when primary HSCs were incubated with ASTX during their activation, α-SMA mRNA and protein levels were markedly decreased, indicating that ASTX prevents the early stage activation of quiescent HSCs.

The foregoing experiments detailed in Example 1, using both LX-2 and primary mouse HSCs, provide the first evidence that ASTX possesses anti-fibrogenic properties and prevents the early stage activation of quiescent HSCs. The inhibitory effect of ASTX on the expression of pro-fibrogenic mediators is attributable in part to the inhibition of the TGFβ1-Smad3 signaling pathway as evidenced by the attenuation of TGFβ1-induced Smad3 phosphorylation and nuclear translocation by ASTX.

### Example 2

### Astaxanthin Prevents and Reverses the Activation of Hepatic Stellate Cells Via the Modulation of Histone Deacetylase 9

### Materials and Methods

### Cell Culture

Mouse primary HSCs were isolated from C57BL/6J, nuclear erythroid 2-related factor 2 (NRF2) knockout (Nrf2−/−), and wild-type mice using a pronase/collagenase digestion method as described herein Example 1. The cells were then cultured on untreated petri dishes (BD Falcon, Franklin Lakes, N.J.), and maintained in a low-glucose DMEM medium supplemented with 10% FBS, 4 mM L-glutamine, and P/S (100 U/100 μg per mL). Human primary HSCs (Zen-Bio, Research Triangle Park, N.C.) were maintained in a low-glucose DMEM medium containing 2% FBS, 4 mM L-glutamine, 1× nonessential amino acids, 1× vitamins, and P/S (100 U/100 μg per mL). The cells were seeded on a collagen-coated plate for experiments (Sarstedt, Newton, N.C.). LX-2 cells, kindly provided by Dr. Scott Friedman at the Icahn School of Medicine at Mount Sinai (New York, N.Y.), were maintained in a low-glucose DMEM supplemented with 2% FBS, 4 mM L-glutamine, and P/S (100 U/100 μg per mL). All cells were cultured in a 37° C. humidified cell culture chamber with 5% CO2. Cell culture supplies were purchased from HyClone unless stated otherwise (Thermo Scientific, Logan, Utah).

### ASTX Treatment

ASTX, provided by Fuji Chemical Industry Co., Ltd. (Toyama, Japan), was prepared in DMSO as described herein in Example 1. After plated on an uncoated dish, mouse qHSCs were incubated with 25 μM ASTX during activation. aHSCs, which were prepared by culturing qHSCs on an uncoated dish for 6 d, were incubated with 25 μM ASTX for 2 or 4 more days. Also, cells were incubated with 3 μM tert-butyl hydrogen peroxide (tBHP, Thermo Scientific) for 12 h with or without 25 μM ASTX. Medium containing ASTX was replenished daily and DMSO vehicle control was always run in parallel. Human primary HSC were treated with 25 μM ASTX for 24 h, after which they were activated by 3 ng/mL of TGFβ1 (Peprotech, Rocky Hill, N.J.) for 24 h.

### Human Liver Specimens

Normal human liver specimens and human livers with primary biliary cirrhosis (PBC) were obtained from Liver Tissue Procurement and Distribution System (Minneapolis, Minn.).

Quantitative Real-Time PCR (qRT-PCR)

Total RNA extraction, cDNA synthesis, and qRT-PCR were conducted using a Bio-Rad CFX96 Real-Time system (Bio-Rad, Hercules, Calif.) as previously described (Id.). Primer sequences will be available upon request.

### Western Blot Analysis

Whole cell lysates and human tissue protein extracts were prepared and Western blot analysis were performed as described (Id.) using antibodies against α-SMA (Sigma, St. Louis, Mo.), HDAC3 (Santa Cruz Biotechnology, Santa Cruz, Calif.), HDAC4 (Santa Cruz Biotechnology), HDAC9 (Abcam, Cambridge, Mass.; Novus Biologicals, Littleton, Colo.), Col1A1 (Sigma). β-tubulin (Santa Cruz) or glyceraldehyde 3-phosphate dehydrogenase (GAPDH) (Santa Cruz) was used as a loading control.

### ROS Measurement

Mouse primary qHSCs were seeded on an uncoated black 24-well μ-Plate (Ibidi, Verona, Wis.). At day 6 of activation, cellular ROS levels were measured using dichlorofluorescin (Sigma-Aldrich, St. Louis, Mo.) as described (Id.). The cells were then fixed with 4% formaldehyde for 10 min, and incubated with 0.1% CellTag 700 stain (Li—COR, Lincoln, Nebr.), a fluorescent stain, for cell number normalization, for 1 h. Fluorescent signals were obtained by an Odyssey® CLx Infrared Imaging System (Li—COR) and a Li—COR Image Studio software. Data were expressed as relative fluorescent intensity divided by total cell stain signal.

Small Interference RNA (siRNA) Transfection

LX-2 cells were transfected with either Silencer Negative Control scrambled siRNA as described herein in Example 1. Subsequently, the cells were pretreated with 25 μM ASTX for 12 h, and then activated by 3 ng/mL TGFβ1 for 12 h.

### Statistical Analysis

One-way analysis of variance with Newman Keul's pairwise comparison, or unpaired t-test was used to determine significant differences between groups by GraphPad Prism6 (GraphPad Software, La Jolla, Calif.). P values less than 0.05 were considered significant and all values were presented as mean±SEM.

### Results

ASTX Inhibited the Activation of qHSCs

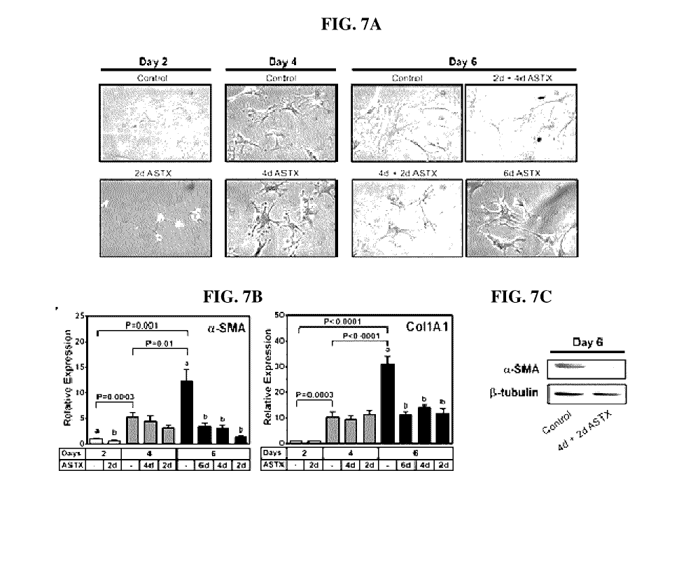

To investigate the effect of ASTX on the activation of qHSCs, mouse primary HSCs were cultured on plastic substratum for 6 d in the absence or presence of ASTX. At days 2 and 4, no noticeable differences in cell morphology were observed between control and ASTX-treated cells. However, at day 6, control cells displayed stretched, myofibroblast-like appearance with few cytoplasmic lipid droplets whereas the cells treated with ASTX for 2-6 days displayed more lipid droplets than controls (FIG. 7A). The expression of α-SMA and Col1A1 was markedly induced during activation in controls, whereas ASTX significantly decreased their mRNA (FIG. 7B). Protein levels of α-SMA were also noticeably decreased when HSCs were treated with ASTX for 2 days during activation (FIG. 7C).

### ASTX Prevented Cellular ROS Accumulation Independent of NRF2 in HSCs

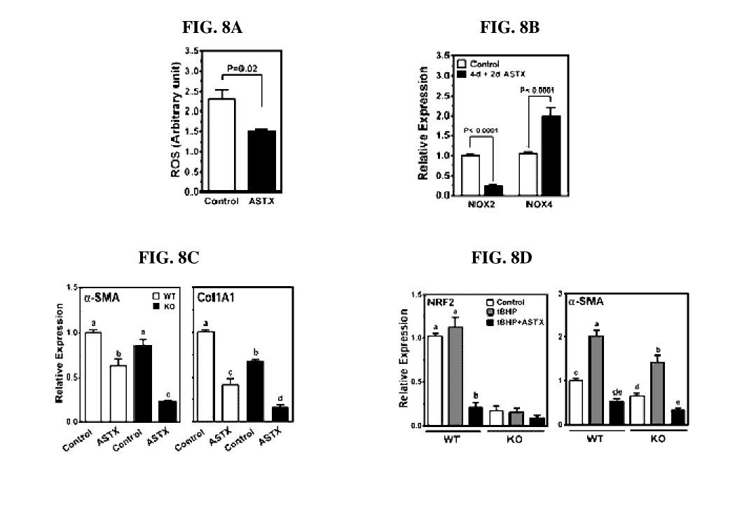

As ROS can activate HSCs, ASTX was evaluated to determine if it can reduce cellular ROS levels. ASTX treatment for 6 d during the activation of mouse primary HSC significantly lowered cellular ROS levels (FIG. 8A). NADPH oxidase 2 (NOX2) and NOX4 have been shown to produce ROS in HSCs, activating the cells. When ASTX was added at day 4 for 2 d during HSC activation, ASTX significantly increased NOX4 mRNA but decreased NOX2 mRNA (FIG. 8B).

The inhibitory action of ASTX in HSC activation mediated via NRF2 was also determined. When HSCs isolated from wild-type and Nrf2^{-/-} mice were activated for 6 d, α-SMA mRNA was not significantly different between HSCs from wild-type and Nrf2^{-/-} mice, while there was a significantly lower Col1A1 in Nrf2^{-/-} HSCs than wild-type (FIG. 8C). Both in wild-type and Nrf2^{-/-} HSCs, ASTX was able to decrease α-SMA and Col1A1 mRNA. As NRF2 may be required when HSCs are under oxidative stress, HSCs from wild-type and Nrf2^{-/-} mice were treated with tBHP at day 6 for 12 h in the presence or absence of ASTX. In both wild-type and Nrf2^{-/-} HSCs, tBHP significantly increased α-SMA expression, which was attenuated by ASTX, and NRF2 expression was significantly decreased by ASTX in wild-type HSCs (FIG. 8D).

ASTX Reverted aHSCs to iHSCs

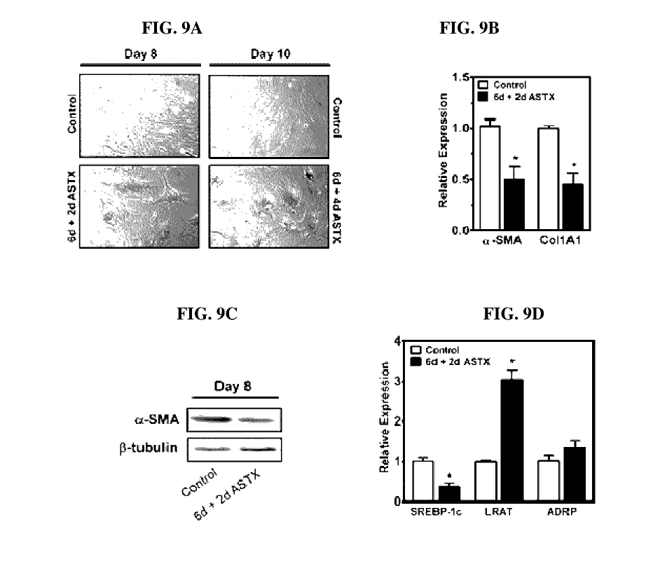

To examine if ASTX can revert aHSCs to iHSCs, aHSCs were treated with ASTX for 2 or 4 d. While control aHSCs displayed a myofibroblast-like appearance, there was reappearance of cytoplasmic lipid droplets in the ASTX-treated HSCs (FIG. 9A). Furthermore, ASTX significantly decreased α-SMA and Col1A1 mRNA (FIG. 9B) and α-SMA protein (FIG. 9C).

As lipid droplets reappeared when aHSCs were incubated with ASTX, the ability of ASTX to alter the expression of genes that facilitate lipid droplet formation was examined. ASTX significantly decreased the mRNA levels of sterol regulatory element binding protein 1c (SREBP1c), a transcriptional factor critical for lipogenesis, but markedly increased lecithin retinol acyltransferase (LRAT) mRNA, an enzyme that esterifies retinol to retinyl esters (FIG. 9D). There was no significant difference in mRNA levels of adipocyte differentiation-related protein (ADRP), a lipid droplet surface protein, between control and ASTX-treated cells.

### ASTX Repressed the Expression of HDAC9 and Myocyte Enhancer Factor 2 (MEF2) in HSCs

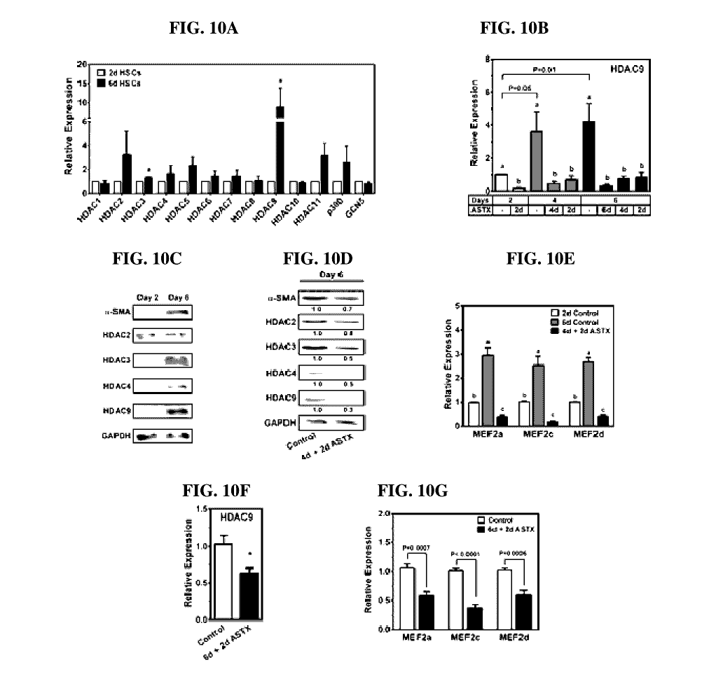

HDACs are a class of enzymes that remove acetyl groups from lysine residues in histones. HDACs, particularly class II HDACs, i.e., HDAC4, 5, 6, 7, 9, and 10, have been suggested to play a critical role in the activation of HSCs. To gain insight into a potential role of HDACs in the modulation of HSC activation by ASTX, alterations in mRNA expression of all 11 classical HDACs were examined during HSC activation. The expression of histone acetyltransferases (HATs), including p300 and general control non-repressible 5, which acetylate histones was measured. While there was a significant, but slight, increase in HDAC3 mRNA, HDAC9 mRNA was increased by ˜10 fold in aHSCs compared with qHSCs (FIG. 10A). ASTX, however, significantly decreased the HDAC9 induction during their activation of qHSCs (FIG. 10B). HDAC9 protein was markedly increased in aHSCs compared with qHSCs, whereas HDAC3 and 4 protein levels were also higher in aHSCs than qHSCs, although their mRNA levels were either increased or remained unchanged during activation (FIG. 10C).

When ASTX was added at day 4 for 2 d during HSC activation, it decreased HDAC3 and 4 protein levels by ˜50% and HDAC9 by ˜70%, with HDAC2 minimally altered (FIG. 10D). The expression of MEF2, a transcription factor, known to induce HDAC9 expression was also measured. MEF2a, 2c and 2d mRNA levels were significantly higher in aHSCs than qHSCs, and ASTX significantly attenuated their induction (FIG. 10E). MEF2b was not detected in qHSCs and aHSCs (data not shown). To further evaluate if the inactivation of aHSCs by ASTX is also mediated by HDAC9, the expression of HDAC9 in aHSCs incubated with or without ASTX for 2 d was also determined. ASTX significantly repressed the mRNA levels of HDAC9 (FIG. 10F) and MEF2 isoforms (FIG. 10G).

### ASTX Repressed Basal and TGFβ1-Induced Expression of Fibrogenic Genes in Human Primary HSCs

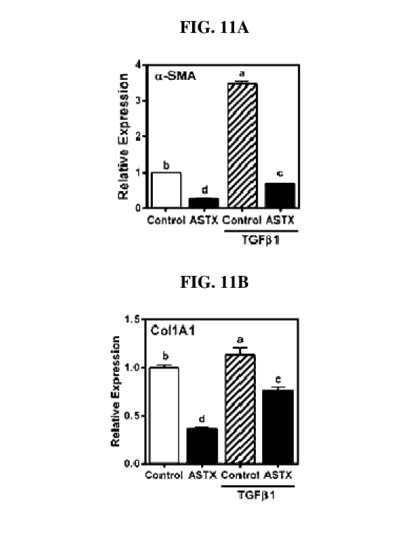

Human relevance of an anti-fibrogenic effect of ASTX was evaluated using human primary HSCs. TGFβ1 significantly increased α-SMA and Col1A1 mRNA levels, but ASTX attenuated the TGFβ1-mediated induction as well as the basal levels of the genes (FIGS. 11A and 11B).

HDAC9 Expression was Significantly Higher in Human PBC Livers than Normal Livers

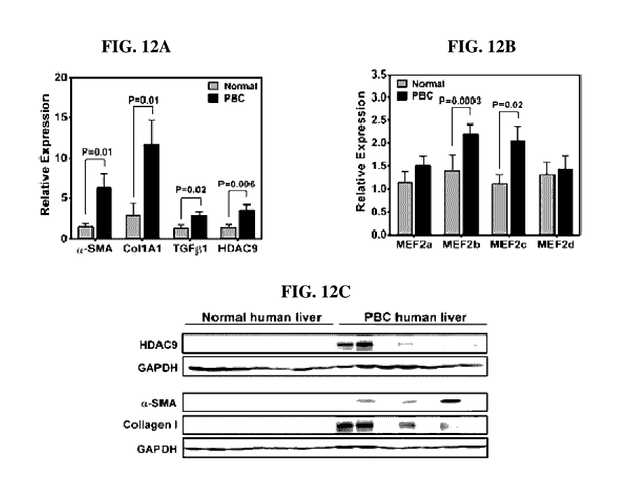

PBC is a chronic liver disease that causes destruction of intrahepatic bile ducts. In the PBC human livers, mRNA levels of α-SMA, Col1A1, TGFβ1 and HDAC9 were markedly increased (FIG. 12A) with concomitant increases in MEF2b and 2c (FIG. 12B), compared to normal human livers. The protein levels of HDAC9, α-SMA, and Collagen I were also significantly increased in human PBC livers (FIG. 12C).

### HDAC9 Knockdown Attenuated TGFβ1-Induced Fibrogenic Gene Expression in LX-2 Cells

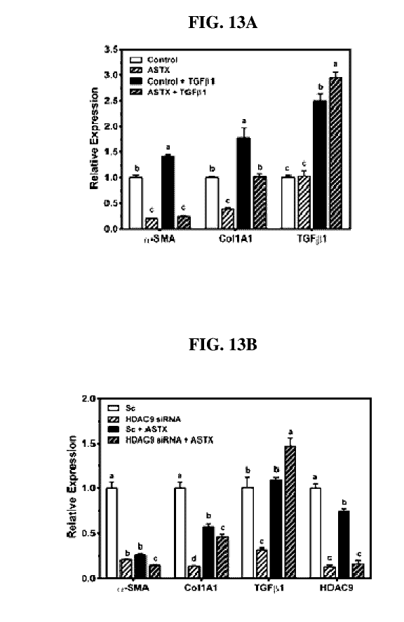

As shown in Example 1, TGFβ1 significantly increased the expression of α-SMA, Col1A1, and TGFβ1 (FIG. 13A). ASTX not only repressed the basal levels of α-SMA and Col1A1 expression, but also attenuated their expression induced by TGFβ1 (FIG. 13A). To determine if HDAC9 mediates TGFβ1-induced fibrogenic response, HDAC9 was knocked down in LX-2 cells by ˜70-80%. Expression of α-SMA, Col1A1 and TGFβ1 was markedly lower in the LX-2 cells transfected with HDAC9 siRNA than in controls (FIG. 13B).

### Discussion

The foregoing results presented herein in Example 2 extend the finding that ASTX has anti-fibrogenic effects in LX-2 cells to human primary HSCs. Also, using mouse primary HSCs, the role of ASTX in the regulation of activation and inactivation of HSCs was evaluated herein. As shown in Example 2, ASTX not only prevented the activation of qHSC but also facilitated the reversion of aHSC to an inactivated state. The effects of ASTX were attributable to its inhibitory action on the expression of HDAC9 and its known transcriptional regulator MEF2. Importantly, using human PBC livers, HDAC9 and MEF2 were shown to play a role in human liver diseases.

In a healthy liver, qHSCs store retinoids in the cytosol and maintain an equilibrium between ECM production and degradation. Upon liver injury, HSCs transdifferentiate to myofibroblasts and express α-SMA and ECM proteins at high abundance. When qHSCs are plated on an uncoated plastic surface, they undergo “spontaneous activation” and are fully activated within 7 days with similar characteristics of HSCs shown in vivo during liver injury. The foregoing results show that ASTX inhibits this activation process, as demonstrated by the attenuated loss of lipid droplets and expression of fibrogenic markers. Without being bound by any one particular theory, one possible explanation may relate to a potent antioxidant property of ASTX. ROS, produced by damaged hepatocytes, Kupffer cells and aHSCs, can activate HSCs and increase ECM production. Herein, ASTX decreased cellular ROS accumulation in HSCs.

Further, NOX2 and NOX4 are major superoxide-producing enzymes in HSCs, and their expression is induced during HSC activation in vitro. As shown in Example 2, ASTX increased NOX4 mRNA, while repressing NOX2 mRNA, suggesting that the decrease in cellular ROS by ASTX is mediated in part by diminished expression of NOX2, but not NOX4.

As a master transcriptional regulator of endogenous antioxidant system, NRF2 senses cellular oxidative stress and induces antioxidant enzymes and glutamate-cysteine ligase catalytic subunit (GCLc). GCLc expression has been shown to decrease during the activation of rat primary HSCs and increases when HSCs are inactivated. Therefore, the potential role of NRF2 in HSC activation was determined. In Example 2, the expression of α-SMA and Col1A1 was not affected in Nrf2^{-/-} HSCs relative to wild-type HSCs regardless of the presence of tBHP. ASTX was shown to inhibit α-SMA and Col1A1 expression similarly in both wild-type and Nrf2^{-/-} HSCs, which may suggest that the repression of the fibrogenic genes by ASTX is likely independent of NRF2. Herein, Nrf2^{-/-} HSCs were observed to show less α-SMA expression regardless the presence or absence of tBHP than wild-type counterparts. There are no published reports that address a direct effect of NRF2 on the fibrogenic gene expression in aHSCs. The data presented herein, however, suggest that, at least in mouse primary HSCs activated on plastic substratum, deficiency of NRF2 does not impact HSC activation.

Recent evidence shows that liver fibrosis is reversible when fibrogenic stimulants are cleared. The reversal of liver fibrosis is accompanied by a reduction in the number of aHSCs by apoptosis or inactivation of aHSCs to a nearly quiescent phenotype, i.e., iHSCs. iHSCs are characterized by the presence of cytosolic lipid droplets and diminished expression of fibrogenic markers, but iHSCs differ from qHSCs because they are more sensitive to fibrogenic stimulus than qHSCs. As shown in Example 2, when aHSCs were incubated with ASTX, cytoplasmic lipid droplets reappeared with a concomitant decrease in α-SMA and ColA1 mRNA, suggesting that aHSCs were reverted to iHSCs. Some studies have suggested that lipogenic genes, such as SREBP-1c and peroxisome proliferator-activated receptor γ, contribute to the inactivation of aHSCs. However, the results presented herein suggest a decrease in SREBP-1c mRNA when aHSCs were incubated with ASTX with no significant change in the expression of ADRP, a surface protein present in lipid droplets in HSCs. Instead, ASTX significantly increased the expression of LRAT. LRAT-deficient mice completely lack lipid droplets in HSCs, suggesting a critical role of LRAT in lipid droplet formation in HSCs, although the LRAT-deficient mice did not have elevated fibrogenesis. Moreover, increased LRAT expression in mice has been shown to increase hepatic retinoid storage and inhibit liver fibrosis. Therefore, without being bound to any one particular theory ASTX may facilitate the reappearance of lipid droplets via the induction of LRAT expression.

Epigenetic modifications, particularly histone acetylation, has been shown to play crucial roles in HSC activation and liver fibrosis. Pan-HDAC inhibitors, such as trichostatin A and valproic acid, are known to be potent inhibitors of HSC activation both in vitro and in vivo. Using inhibitors specific for Class II HDACs, studies have shown that class II HDACs play important roles in HSC activation. The results presented in Example 2 demonstrate that HDAC9 mRNA and protein levels in aHSCs were significantly elevated compared to qHSCs, while HDAC3 and HDAC4 protein levels were also markedly increased after HSC activation although their mRNA levels were minimally induced. Interestingly, ASTX decreased HDAC3, 4, and 9 protein levels by ˜50-70%. The data suggest that ASTX decreased HDAC9 expression at the transcriptional levels, whereas HDAC3 and HDAC4 are likely regulated post-transcriptionally. It has been shown that MEF2a, 2c and 2d are induced during HSC activation while decreased when HSCs are inactivated, and that MEF2 knockdown down-regulates α-SMA and Col1A1 expression in primary rat HSCs. The foregoing results show that the expression of MEF2a, 2c and 2d was elevated by HSC activation but their induction was markedly attenuated by ASTX in parallel with repressed HDAC9 expression. Of importance is that the expression of both HDAC9 mRNA and protein were markedly increased in PBC human livers. Likewise, the expression of MEF2b and 2c was also increased relative to normal livers. Furthermore, when HDAC9 was knocked down in LX-2 cells by ˜70% using siRNA, the expression of TGFβ1-induced α-SMA, Col1A1 and TGFβ1 was significantly attenuated, supporting that HDAC9 may play a critical role in HSC activation. Therefore, the data presented herein suggest that ASTX may inhibit MEF2 expression or transcriptional activity, consequently repressing HDAC9 expression and HSC activation. Furthermore, protein levels of HDAC3, which belongs to Class I HDACs, were increased by HSC activation but decreased by ASTX similarly to HDAC4. Class I HDACs are also known to play a role in HSC activation. Therefore, the repression of HDAC3 and 4 expression by ASTX at the post-transcriptional levels may also play a role in the anti-fibrogenic action of ASTX.

In conclusion, the results of the foregoing experiments exemplified in Example 2 suggest that ASTX inhibits the activation of qHSCs and also reverts aHSCs to an inactivate state. This anti-fibrogenic effect of ASTX may be mediated via the pathways regulated by MEF2/HDAC9 axis. Although further work is needed to elucidate the underlying mechanism, the present study provides novel evidence that ASTX is anti-fibrogenic and inhibits HSC activation. These findings demonstrate that ASTX may be used as a natural anti-fibrogenic agent.

1. A method of reversing fibrosis in a subject comprising administering to the subject an effective amount of astaxanthin (ASTX), a pharmaceutically acceptable salt thereof, or a pharmaceutical composition comprising astaxanthin (ASTX) and a pharmaceutically acceptable excipient, thereby reversing fibrosis in the subject.

2. The method of claim 1, wherein the subject is suffering from a fibrotic disease.

3. The method of claim 2, wherein the fibrotic disease is one or more of aberrant wound healing, acute interstitial pneumonitis, arthrofibrosis, asthma, atherosclerosis, bone-marrow fibrosis, cardiac fibrosis, chronic kidney disease, cirrhosis of gallbladder, cirrhosis of liver, colloid and hypertrophic scar, Crohn's disease, cryptogenic organizing pneumonia, cystic fibrosis, desquamative interstitial pneumonia, diffuse parenchymal lung disease, Dupuytren's contracture, endomyocardial fibrosis, fibrosis as a result of Graft-Versus-Host Disease (GVHD), glomerulonephritis, idiopathic interstitial fibrosis, interstitial lung disease, interstitial pneumonitis, keloid scar, hypertrophic scar, lymphocytic interstitial pneumonia, morphea, multifocal fibrosclerosis, muscle fibrosis, myelofibrosis, nephrogenic systemic fibrosis, nonspecific interstitial pneumonia, organ transplant fibrosis, pancreatic fibrosis, Peyronie's disease, pulmonary fibrosis, renal fibrosis, respiratory bronchiolitis, retroperitoneal fibrosis, scarring after surgery, scleroderma, or subepithelial fibrosis.

4. The method of claim 2, wherein the fibrotic disease is one or more of fibrosis of the bone marrow, fibrosis of the gallbladder, fibrosis of the heart, fibrosis of the liver, fibrosis of the lung, fibrosis of the kidney, fibrosis of the muscle, fibrosis of the pancreas, fibrosis of the penis, or fibrosis of the uterus.

5. The method of claim 4, wherein the fibrotic disease is fibrosis of the liver.

6. The method of claim 1, wherein the subject is a human.

7. The method of claim 1, wherein the subject suffers from at least one of chronic Hepatitis B, Hepatitis C, non-alcoholic steatophepatitis (NASH), alcoholic liver disease, a metabolic liver disease, Wilson's disease, hemochromatosis, or biliary obstruction.

8. The method of claim 1, wherein the ASTX exhibits an activity selected from the group consisting of inhibiting fibrosis, reversing activation of hepatic stellate cells (HSC), preventing activation of hepatic stellate cells (HSC), inhibiting TGFβ1 signaling, inhibiting activation of the Smad3 pathway in hepatic stellate cells (HSC), inhibiting HDAC9 expression, inhibiting cellular reactive oxygen species (ROS) accumulation, inhibiting expression of myocyte enhancer factor 2 (MEF2), inhibiting basal expression of fibrogenic genes, and inhibiting TGFβ1-induced expression of fibrogenic genes.

9. A method of reversing a fibrotic disease in a subject comprising administering to the subject an effective amount of astaxanthin (ASTX), a pharmaceutically acceptable salt thereof, or a pharmaceutical composition comprising astaxanthin (ASTX) and a pharmaceutically acceptable excipient, thereby reversing the fibrotic disease in the subject

10. The method of claim 9, wherein the fibrotic disease is a chronic condition.

11. The method of claim 9, wherein the fibrotic disease is one or more of aberrant wound healing, acute interstitial pneumonitis, arthrofibrosis, asthma, atherosclerosis, bone-marrow fibrosis, cardiac fibrosis, chronic kidney disease, cirrhosis of gallbladder, cirrhosis of liver, colloid and hypertrophic scar, Crohn's disease, cryptogenic organizing pneumonia, cystic fibrosis, desquamative interstitial pneumonia, diffuse parenchymal lung disease, Dupuytren's contracture, endomyocardial fibrosis, fibrosis as a result of Graft-Versus-Host Disease (GVHD), glomerulonephritis, idiopathic interstitial fibrosis, interstitial lung disease, interstitial pneumonitis, keloid scar, hypertrophic scar, lymphocytic interstitial pneumonia, morphea, multifocal fibrosclerosis, muscle fibrosis, myelofibrosis, nephrogenic systemic fibrosis, nonspecific interstitial pneumonia, organ transplant fibrosis, pancreatic fibrosis, Peyronie's disease, pulmonary fibrosis, renal fibrosis, respiratory bronchiolitis, retroperitoneal fibrosis, scarring after surgery, scleroderma, or subepithelial fibrosis.

12. The method of claim 9, wherein the fibrotic disease is one or more of fibrosis of the bone marrow, fibrosis of the gallbladder, fibrosis of the heart, fibrosis of the liver, fibrosis of the lung, fibrosis of the kidney, fibrosis of the muscle, fibrosis of the pancreas, fibrosis of the penis, or fibrosis of the uterus.

13. The method of claim 12, wherein the fibrotic disease is fibrosis of the liver.

14. The method of claim 9, wherein the subject is a human.

15. The method of claim 13, wherein the subject suffers from at least one of chronic Hepatitis B, Hepatitis C, non-alcoholic steatophepatitis (NASH), alcoholic liver disease, a metabolic liver disease, Wilson's disease, hemochromatosis, or biliary obstruction.

16. The method of claim 9, wherein the ASTX exhibits an activity selected from the group consisting of reversing fibrosis, inhibiting fibrosis, reversing activation of hepatic stellate cells (HSC), preventing activation of hepatic stellate cells (HSC), inhibiting TGFβ1 signaling, inhibiting activation of the Smad3 pathway in hepatic stellate cells (HSC), inhibiting HDAC9 expression, inhibiting cellular reactive oxygen species (ROS) accumulation, inhibiting expression of myocyte enhancer factor 2 (MEF2), inhibiting basal expression of fibrogenic genes, and inhibiting TGFβ1-induced expression of fibrogenic genes.

17. A method of reversing the activation of activated hepatic stellate cells (aHSCs), comprising contacting the activated hepatic stellate cells with astaxanthin (ASTX), or a pharmaceutically acceptable salt thereof, thereby reversing the activation of activated hepatic stellate cells.

18. The method of claim 17, wherein the activated hepatic stellate cells are murine or human primary hepatic stellate cells (HSCs).

19. The method of claim 17, wherein the activated hepatic stellate cells (aHSCs) are reversed to quiescent (qHSCs) or inactive hepatic stellate cells (iHSCs).

20. The method of claim 17, wherein the ASTX exhibits an activity selected from the group consisting of inhibiting TGFβ1 signaling, inhibiting activation of the Smad3 pathway in hepatic stellate cells (HSC), inhibiting HDAC9 expression, inhibiting cellular reactive oxygen species (ROS) accumulation, inhibiting expression of myocyte enhancer factor 2 (MEF2), inhibiting basal expression of fibrogenic genes, and inhibiting TGFβ1-induced expression of fibrogenic genes.
